SEQUENCE LISTING (24 sequences)

SEQ ID NO: 1 - DNA, 1796 bp, Homo sapiens
  CDS (4)..(1509)
  mat_peptide (46)..()
gcgatgtcgctcgtgctgctaagcctggccgcgctgtgcaggagcgccgtaccccgagagccgaccgttcaatgtggctctgaaactgggccatctccagagtggatgctacaacatgatctaatcccgggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctatatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagctttaattgaaaactgtaaccattttgataatgcaacaataaagcatcttcagcc

SEQ ID NO: 2 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 3 - DNA, 637 bp, Mus musculus
  CDS (1)..(210)
gatttcagcagccagacgcatctgcacaaatacctggaggtctatcttgggggagcagacctcaaaggcgactataatgccctgagtgtctgcccccaatatcatctcatgaaggacgccacagctttccacacagaacttctcaaggctacgcagagcatgtcagtgaagaaacgctcacaagcctgccatgatagctgttcacccttgtagtccacccgggggaatagagactctgaagccttcctactctcccttccagtgacaaatgctgtgtgacgactctgaaatgtgtgggagaggctgtgtggaggtagtgctatgtacaaacttgctttaaaactggagtttgcaaagtcaacctgagcatacacgcctgaggctagtcattggctggatttatgaagacaacacagttacagacaataatgagtgggacctacatttgggatatacccaaagctgggtaatgattatcactgagaaccacgcactctggccatgaggtaatacggcacttccctgtcaggctgtctgtcaggttgggtctgtcttgcactgcccatgctctatgctgcacgtagaccgttttgtaacattttaatctgttaatgaataatccgtttgggaggctctc

SEQ ID NO: 4 - protein, 70 aa, Mus musculus
DFSSQTHLHKYLEVYLGGADLKGDYNALSVCPQYHLMKDATAFHTELLKATQSMSVKKRSQACHDSCSPL

SEQ ID NO: 5 - DNA, 2308 bp, Homo sapiens
  misc_feature (140)..(140): unknown amino
gagtcaggactcccaggacagagagtgcacaaactacccagcacagccccctccgccccctctggaggctgaagagggattccagcccctgccacccacagacacgggctgactggggtgtctgccccccttgggggcanccacagggcctcaggcctgggtgccacctggcactagaagatgcctgtgccctggttcttgctgtccttggcactgggccgaagccagtggatcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgacttaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaatgtcactgtggacgtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggacntccgcgccgggacgcggggtgggaccaggggcgggacctggggcgggggacgggacttaaataaaggcagacgctg

SEQ ID NO: 6 - protein, 703 aa, Homo sapiens
MPVPWFLLSLALGRSQWILSLERLVGPQDATHCSPGLSCRLWDSDILCLPGDIVPAPGPVLAPTHLQTELVLRCQKETDCDLCLRVAVHLAVHGHWEEPEDEEKFGGAADLGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTSAPGRGVGPGAGPGAGDGT

SEQ ID NO: 7 - DNA, 2314 bp, Mus musculus
  CDS (199)..(2292)
  mat_peptide (259)..()
ccaaatcgaaagcacgggagctgatactgggcctggagtccaggctcactggagtggggaagcatggctggagaggaattctagcccttgctctctcccagggacacggggctgattgtcagcaggggcgaggggtctgcccccccttgggggggcaggacggggcctcaggcctgggtgctgtccggcacctggaagatgcctgtgtcctggttcctgctgtccttggcactgggccgaaaccctgtggtcgtctctctggagagactgatggagcctcaggacactgcacgctgctctctaggcctctcctgccacctctgggatggtgacgtgctctgcctgcctggaagcctccagtctgccccaggccctgtgctagtgcctacccgcctgcagacggagctggtgctgaggtgtccacagaagacagattgcgccctctgtgtccgtgtggtggtccacttggccgtgcatgggcactgggcagagcctgaagaagctggaaagtctgattcagaactccaggagtctaggaacgcctctctccaggcccaggtggtgctctccttccaggcctaccccatcgcccgctgtgccctgctggaggtccaggtgcccgctgacctggtgcagcctggtcagtccgtgggttctgcggtatttgactgtttcgaggctagtcttggggctgaggtacagatctggtcctacacgaagcccaggtaccagaaagagctcaacctcacacagcagctgcctgactgcaggggtcttgaagtccgggacagcatccagagctgctgggtcctgccctggctcaatgtgtctacagatggtgacaatgtccttctgacactggatgtctctgaggagcaggactttagcttcttactgtacctgcgtccagtcccggatgctctcaaatccttgtggtacaaaaacctgactggacctcagaacattactttaaaccacacagacctggttccctgcctctgcattcaggtgtggtcgctagagccagactctgagagggtcgaattctgccccttccgggaagatcccggtgcacacaggaacctctggcacatagccaggctgcgggtactgtccccaggggtatggcagctagatgcgccttgctgtctgccgggcaaggtaacactgtgctggcaggcaccagaccagagtccctgccagccacttgtgccaccagtgccccagaagaacgccactgtgaatgagccacaagatttccagttggtggcaggccaccccaacctctgtgtccaggtgagcacctgggagaaggttcagctgcaagcgtgcttgtgggctgactccttggggcccttcaaggatgatatgctgttagtggagatgaaaaccggcctcaacaacacatcagtctgtgccttggaacccagtggctgtacaccactgcccagcatggcctccacgagagctgctcgcctgggagaggagttgctgcaagacttccgatcacaccagtgtatgcagctgtggaacgatgacaacatgggatcgctatgggcctgccccatggacaagtacatccacaggcgctgggtcctagtatggctggcctgcctactcttggctgcggcgcttttcttcttcctccttctaaaaaaggaccgcaggaaagcggcccgtggctcccgcacggccttgctcctccactccgccgacggagcgggctacgagcgcctggtgggagcactggcgtccgcgttgagccagatgccactgcgcgtggccgtggacctgtggagccgccgcgagctgagcgcgcacggagccctagcctggttccaccaccagcgacgccgtatcctgcaggagggtggcgtggtaatccttctcttctcgcccgcggccgtggcgcagtgtcagcagtggctgcagctccagacagtggagcccgggccgcatgacgccctcgccgcctggctcagctgcgtgctacccgatttcctgcaaggccgggcgaccggccgctacgtcggggtctacttcgacgggctgctgcacccagactctgtgccctccccgttccgcgtcgccccgctcttctccctgccctcgcagctgccggctttcctggatgcactgcagggaggctgctccacttccgcggggcgacccgcggaccgggtggaacgagtgacccaggcgctgcggtccgccctggacagctgtacttctagctcggaagccccaggctgctgcgaggaatgggacctgggaccctgcactacactagaataaaagccgatacagtattcct

SEQ ID NO: 8 - protein, 698 aa, Mus musculus
MPVSWFLLSLALGRNPVVVSLERLMEPQDTARCSLGLSCHLWDGDVLCLPGSLQSAPGPVLVPTRLQTELVLRCPQKTDCALCVRVVVHLAVHGHWAEPEEAGKSDSELQESRNASLQAQVVLSFQAYPIARCALLEVQVPADLVQPGQSVGSAVFDCFEASLGAEVQIWSYTKPRYQKELNLTQQLPDCRGLEVRDSIQSCWVLPWLNVSTDGDNVLLTLDVSEEQDFSFLLYLRPVPDALKSLWYKNLTGPQNITLNHTDLVPCLCIQVWSLEPDSERVEFCPFREDPGAHRNLWHIARLRVLSPGVWQLDAPCCLPGKVTLCWQAPDQSPCQPLVPPVPQKNATVNEPQDFQLVAGHPNLCVQVSTWEKVQLQACLWADSLGPFKDDMLLVEMKTGLNNTSVCALEPSGCTPLPSMASTRAARLGEELLQDFRSHQCMQLWNDDNMGSLWACPMDKYIHRRWVLVWLACLLLAAALFFFLLLKKDRRKAARGSRTALLLHSADGAGYERLVGALASALSQMPLRVAVDLWSRRELSAHGALAWFHHQRRRILQEGGVVILLFSPAAVAQCQQWLQLQTVEPGPHDALAAWLSCVLPDFLQGRATGRYVGVYFDGLLHPDSVPSPFRVAPLFSLPSQLPAFLDALQGGCSTSAGRPADRVERVTQALRSALDSCTSSSEAPGCCEEWDLGPCTTLE

SEQ ID NO: 9 - DNA, 2786 bp, Homo sapiens
  misc_feature (8)..(8): unknown amino
cccacgcntccgggccagcagcgggcggccggggcgcagagaacggcctggctgggcgagcgcacggccatggccccgtggctgcagctctgctccgtcttctttacggtcaacgcctgcctcaacggctcgcagctggctgtngccgctggcgggtccggccgcgcgcngggcgccgacacctgtagctggangggagtggggccagccagcagaaacagtgggctgtacaacatcaccttcaaatatgacaattgtaccacctacttgaatccagtggggaagcatgtgattgctgacgcccagaatatcaccatcagccagtatgcttgccatgaccaagtggcagtcaccattctttggtccccaggggccctcggcatcgaattcctgaaaggatttcgggtaatactggaggagctgaagtcggagggaagacagngccaacaactgattctaaaggatccgaagcagntcaacagtagcttcaaaagaactggaatggaatctcaacctttnctgaatatgaaatttgaaacggattatttcgtaaggttgtccttttccttcattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtaagcaggagcaaactacagagatgaccagctgcctccttcaaaatgtttctccaggggattatataattgagctggtggatgacactaacacaacaagaaaagtgatgcattatgccttaaagccagtgcactccccgtgggccgggcccatcagagccgtggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggtatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcggctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaaacgaaagagtctaagcattgccactttagctgctgcctccctctgattccccagctcatctccctggttgcatggcccacttggagctgaggtctcatacaaggatatttggagtgaaatgctggccagtacttgttctcccttgccccaaccctttaccggatatcttgacaaactctccaattttctaaaatgatatggagctctgaaaggcatgtccataaggtctgacaacagcttgccaaatttggttagtccttggatcagagcctgttgtgggaggtagggaggaaatatgtaaagaaaaacaggaagatacctgcactaatcattcagacttcattgagctctgcaaactttgcctgtttgctattggctaccttgatttgaaatgctttgtgaaaaaaggcacttttaacatcatagccacagaaatcaagtgccagtctatctggaatccatgttgtattgcagataatgttctcatttatttttg

SEQ ID NO: 10 - protein, 738 aa, Homo sapiens
  misc_feature (18)..(18): The ′Xaa′ at location 18 stands for Gln, Arg, Pro, or Leu.
MAPWLQLCSVFFTVNACLNGSQLAVAAGGSGRAXGADTCSWXGVGPASRNSGLYNITFKYDNCTTYLNPVGKHVIADAQNITISQYACHDQVAVTILWSPGALGIEFLKGFRVILEELKSEGRQXQQLILKDPKQXNSSFKRTGMESQPXLNMKFETDYFVRLSFSFIKNESNYHPFFFRTRACDLLLQPDNLACKPFWKPRNLNISQHGSDMQVSFDHAPHNFGFRFFYLHYKLKHEGPFKRKTCKQEQTTEMTSCLLQNVSPGDYIIELVDDTNTTRKVMHYALKPVHSPWAGPIRAVAITVPLVVISAFATLFTVMCRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 11 - DNA, 2012 bp, Homo sapiens
  CDS (1)..(1971)
  mat_peptide (70)..()
atggggagctccagactggcagccctgctcctgcctctcctcctcatagtcatcgacctctctgactctgctgggattggctttcgccacctgccccactggaacacccgctgtcctctggcctcccacacggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggtgggcactgggtacaacaggagatgggttcagctgagtggtggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaaggtacaaccatggttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggtgtggcggtcagatgtccagtttgcctggaagcacctcttgtgtccagatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttgagcagagctccaccgcagtcccgggtgtctgcggccgct

SEQ ID NO: 12 - protein, 657 aa, Homo sapiens
MGSSRLAALLLPLLLIVIDLSDSAGIGFRHLPHWNTRCPLASHTEVLPISLAAPGGPSSPQSLGVCESGTVPAVCASICCQVAQVFNGASSTSWCRNPKSLPHSSSIGDTRCQHLLRGSCCLVVTCLRRAITFPSPPQTSPTRDFALKGPNLRIQRHGKVFPDWTHKGMEVGTGYNRRWVQLSGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKVQPWFSFGNSSHVECPHQTGSLTSWNVSMDTQAQQLILHFSSRMHATFSAAWSLPGLGQDTLVPPVYTVSQVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 13 - DNA, 808 bp, Mus musculus
  CDS (78)..(806)
  mat_peptide (147)..()
cagctccgggccaggccctgctgccctcttgcagacaggaaagacatggtctctgcgccctgatcctacagaagctcatggggagccccagactggcagccttgctcctgtctctcccgctactgctcatcggcctcgctgtgtctgctcgggttgcctgcccctgcctgcggagttggaccagccactgtctcctggcctaccgtgtggataaacgttttgctggccttcagtggggctggttccctctcttggtgaggaaatctaaaagtcctcctaaatttgaagactattggaggcacaggacaccagcatccttccagaggaagctgctaggcagcccttccctgtctgaggaaagccatcgaatttccatcccctcctcagccatctcccacagaggccaacgcaccaaaagggcccagccttcagctgcagaaggaagagaacatctccctgaagcagggtcacaaaagtgtggaggacctgaattctcctttgatttgctgcccgaggtgcaggctgttcgggtgactattcctgcaggccccaaggcacgtgtgcgcctttgttatcagtgggcactggaatgtgaagacttgagtagcccttttgatacccagaaaattgtgtctggagggcacactgtagacctgccttatgaattccttctgccctgcatgtgcatagaggcctcctacctgcaagaggacactgtgaggcgcaaaagtgtcccttccagagctggcctgaagcttatggctcagacttctggcagtcaatacgcttcactgactacagccagcac

SEQ ID NO: 14 - protein, 243 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKARVRLCYQWALECEDLSSPFDTQKIVSGGHTVDLPYEFLLPCMCIEASYLQEDTVRRKSVPSRAGLKLMAQTSGSQYASLTTAS

SEQ ID NO: 15 - DNA, 2377 bp, Homo sapiens
  CDS (180)..(1874)
ttttgagcagaggcttcctaggctccgtagaaatttgcatacagcttccacttcctgcttcagagcctgttcttctacttacctgggcccggagaaggtggagggagacgagaagccgccgagagccgactaccctccgggcccagtctgtctgtccgtggtggatctaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccaagaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaaaaaaaaaaaaaaaaaaaaaaagaaaaaaaaaaaaa

SEQ ID NO: 16 - protein, 565 aa, Homo sapiens
MNRSIPVEVDESEPYPSQLLKPIPEYSPEEESEPPAPNIRNMAPNSLSAPTMLHNSSGDFSQAHSTLKLANHQRPVSRQVTCLRTQVLEDSEDSFCRRHPGLGKAFPSGCSAVSEPASESVVGALPAEHQFSFMEKRNQWLVSQLSAASPDTGHDSDKSDQSLPNASADSLGGSQEMVQRPQPHRNRAGLDLPTIDTGYDSQPQDVLGIRQLERPLPLTSVCYPQDLPRPLRSREFPQFEPQRYPACAQMLPPNLSPHAPWNYHYHCPGSPDHQVPYGHDYPRAAYQQVIQPALPGQPLPGASVRGLHPVQKVILNYPSPWDQEERPAQRDCSFPGLPRHQDQPHHQPPNRAGAPGESLECPAELRPQVPQPPSPAAVPRPPSNPPARGTLKTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFIKQGSMNFRFIPVLFPNAKKEHVPTWLQNTHVYSWPKNKKNILLRLLREEEYVAPPRGPLPTLQVVPL

SEQ ID NO: 17 - DNA, 1323 bp, Mus musculus
  CDS (1)..(1026)
caggacctccctgggcctctgaggtccagggaattgccacctcagtttgaacttgagaggtatccaatgaacgcccagctgctgccgccccatccttccccacaggccccatggaactgtcagtactactgccccggagggccctaccaccaccaggtgccacacggccatggctaccctccagcagcagcctaccagcaagtactccagcctgctctgcctgggcaggtccttcctggggcaagggcaagaggcccacgccctgtgcagaaggtcatcctgaatgactccagcccccaagaccaagaagagagacctgcacagagagacttctctttcccgaggctcccgagggaccagctctaccgcccaccatctaatggagtggaagcccctgaggagtccttggaccttcctgcagagctgagaccacatggtccccaggctccatccctagctgccgtgcctagaccccctagcaaccccttagcccgaggaactctaagaaccagcaatttgccagaagaattacggaaagtctttatcacttattctatggacacagccatggaggtggtgaaatttgtgaactttctgttggtgaacggcttccaaactgcgattgacatatttgaggatagaatccggggtattgatatcattaaatggatggagcgctatcttcgagataagacagtgatgataatcgtagcaatcagccccaaatacaaacaggatgtggaaggcgctgagtcgcagctggacgaggacgagcatggcttacatactaagtacattcatcggatgatgcagattgagttcataagtcagggaagcatgaacttcagattcatccctgtgctcttcccaaatgccaagaaggagcatgtgccgacctggcttcagaacactcatgtttacagctggcccaagaataagaaaaacatcctgctgcggctgctcagggaggaagagtatgtggctcctccccgaggccctctgcccacccttcaggtggtacccttgtgacgatggccactccagctcagtgccagcctgttctcacagcattcttctagcggagctggctggtggcacccaggccctggaacacctcttctacagagtcctctgtctcctgagtctgagttgtcctcgctgggcttccagagcttcagtgcctggatgctgcaggtgacagaaacaaacatctatgaccacaaaaactctcatcacttcagctacttttatgagtcggtcagatgctctgtgtccttagaccagtctaaatcatgctcaaataataaaatgattattctttgt

SEQ ID NO: 18 - protein, 342 aa, Mus musculus
QDLPGPLRSRELPPQFELERYPMNAQLLPPHPSPQAPWNCQYYCPGGPYHHQVPHGHGYPPAAAYQQVLQPALPGQVLPGARARGPRPVQKVILNDSSPQDQEERPAQRDFSFPRLPRDQLYRPPSNGVEAPEESLDLPAELRPHGPQAPSLAAVPRPPSNPLARGTLRTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFISQGSMNFRFIPVLFPNAKKEHVPTWLQNTHVYSWPKNKKNILLRLLREEEYVAPPRGPLPTLQVVPL

SEQ ID NO: 19 - protein, 207 aa, Homo sapiens
RKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDW

SEQ ID NO: 20 - protein, 208 aa, Mus musculus
RKVWIVYSADHPLYVEVVLKFAQFLITACGTEVALDLLEEQVISEVGVMTWVSRQKQEMVESNSKIIILCSRGTQAKWKAILGWAEPAVQLRCDHWKPAGDLFTAAMNMILPDFKRPACFGTYVVCYFSGICSERDVPDLFNITSRYPLMDRFEEVYFRIQDLEMFEPGRMHHVRELTGDNYLQSPSGRQLKEAVLRFQEWQTQCPDW

SEQ ID NO: 21 - protein, 190 aa, Caenorhabditis elegans
VKVMIVYADDNDLHTDCVKKLVENLRNCASCDPVFDLEKLITAEIVPSRWLVDQISSLKKFIIVVSDCAEKILDTEASETHQLVQARPFADLFGPAMEMIIRDATHNFPEARKKYAVVRFNYSPHVPPNLAILNLPTFIPEQFAQLTAFLHNVEHTERANVTQNISEAQIHEWNLCASRMMSFFVRNPNW

SEQ ID NO: 22 - protein, 178 aa, Caenorhabditis elegans
FKVMLVCPEVSGRDEDFMMRIADALKKSNNKVVCDRWFEDSKNAEENMLHWVYEQTKIAEKIIVFHSAYYHPRCGIYDVINNFFPCTDPRLAHIALTPEAQRSVPKEVEYVLPRDQKLLEDAFDITIADPLVIDIPIEDVAIPENVPIHHESCDSIDSRNNSKTHSTDSGVSSLSSNS

SEQ ID NO: 23 - DNA, 1107 bp, Homo sapiens
gtgtggcctcaggtataagagcggctgctgccaggtgcatggccaggtgcacctgtgggattgccgccaggtgtgcaggccgctccaagcccagcctgccccgctgccgccaccatgacgctcctccccggcctcctgtttctgacctggctgcacacatgcctggcccaccatgacccctccctcagggggcacccccacagtcacggtaccccacactgctactcggctgaggaactgcccctcggccaggcccccccacacctgctggctcgaggtgccaagtgggggcaggctttgcctgtagccctggtgtccagcctggaggcagcaagccacagggggaggcacgagaggccctcagctacgacccagtgcccggtgctgcggccggaggaggtgttggaggcagacacccaccagcgctccatctcaccctggagataccgtgtggacacggatgaggaccgctatccacagaagctggccttcgccgagtgcctgtgcagaggctgtatcgatgcacggacgggccgcgagacagctgcgctcaactccgtgcggctgctccagagcctgctggtgctgcgccgccggccctgctcccgcgacggctcggggctccccacacctggggcctttgccttccacaccgagttcatccacgtccccgtcggctgcacctgcgtgctgccccgttcagtgtgaccgccgaggccgtggggcccctagactggacacgtgtgctccccagagggcaccccctatttatgtgtatttattggtatttatatgcctcccccaacactacccttggggtctgggcattccccgtgtctggaggacagccccccactgttctcctcatctccagcctcagtagttgggggtagaaggagctcagcacctcttccagcccttaaagctgcagaaaaggtgtcacacggctgcctgtaccttggctccctgtcctgctcccggcttcccttaccctatcactggcctcaggcccccgcaggctgcctcttcccaacctccttggaagtacccctgtttcttaaacaattatttaagtgtacgtgtattattaaactgatgaacacatcc

SEQ ID NO: 24 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

(19) United States
(12) Patent Application Publication
(10) Pub. No.: US 2004/0197306 A1
(43) Pub. Date: Oct. 7, 2004

(54) Mammalian receptor proteins; related reagents and methods

(76) Inventor: Daniel M. Gorman, Palo Alto, CA (US)

Correspondence Address: DNAX RESEARCH, INC., LEGAL DEPARTMENT, 901 CALIFORNIA AVENUE, PALO ALTO, CA, 94304, US

(21) Appl. No.: 10/749,144

(22) Filed: Dec. 29, 2003

Related U.S. Application Data
(63) Continuation-in-part of application No. 09/863,818, filed on May 23, 2001.
(60) Provisional application No. 60/206,862, filed on May 24, 2000.

(51) Int. Cl. A61K 38/20; A61K 38/17

(52) U.S. Cl. 424/85.2; 514/12

(57) ABSTRACT

Nucleic acids encoding mammalian, e.g., primate, receptors, purified receptor proteins and fragments thereof. Antibodies, both polyclonal and monoclonal, are also provided. Methods of using the compositions for both diagnostic and therapeutic utilities are described.

This filing is a Continuation-In-Part of commonly assigned, co-pending application Ser. No. 09/863,818, filed May 23, 2001, which claims benefit of U.S. Provisional Patent Application No. 60/206,862, filed May 24, 2000, each of which is incorporated herein by reference in its entirety.

### FIELD OF THE INVENTION

The present invention relates to compositions and methods for affecting mammalian physiology, including immune system function. In particular, it provides methods to regulate development and/or the immune system. Diagnostic and therapeutic uses of these materials are also disclosed.

### BACKGROUND OF THE INVENTION

Recombinant DNA technology refers generally to techniques of integrating genetic information from a donor source into vectors for subsequent processing, such as through introduction into a host, whereby the transferred genetic information is copied and/or expressed in the new environment. Commonly, the genetic information exists in the form of complementary DNA (cDNA) derived from messenger RNA (mRNA) coding for a desired protein product. The carrier is frequently a plasmid having the capacity to incorporate cDNA for later replication in a host and, in some cases, actually to control expression of the cDNA and thereby direct synthesis of the encoded product in the host. See, e.g., Sambrook, et al. (1989) Molecular Cloning: A Laboratory Manual, (2nd ed.) vols. 1-3, CSH Press, NY.

For some time, it has been known that the mammalian immune response is based on a series of complex cellular interactions, called the “immune network”. Recent research has provided new insights into the inner workings of this network. While it remains clear that much of the immune response does, in fact, revolve around the network-like interactions of lymphocytes, macrophages, granulocytes, and other cells, immunologists now generally hold the opinion that soluble proteins, known as cytokines, play critical roles in controlling these cellular interactions. Thus, there is considerable interest in the isolation, characterization, and mechanisms of action of cell modulatory factors, an understanding of which will lead to significant advancements in the diagnosis and therapy of numerous medical abnormalities, e.g., immune system disorders.

The immune system of vertebrates consists of a number of organs and several different cell types. Two major cell types include the myeloid and lymphoid lineages. Among the lymphoid cell lineage are B cells, which were originally characterized as differentiating in fetal liver or adult bone marrow, and T cells, which were originally characterized as differentiating in the thymus. See, e.g., Paul (ed.) (1998) Fundamental Immunology (4th ed.) Raven Press, New York; and Thomson (ed.) (1994) The Cytokine Handbook 2nd ed., Academic Press, San Diego. Cytokines mediate cellular activities in a variety of ways. They have been shown to support the proliferation, growth, and/or differentiation of cells, e.g., pluripotential hematopoietic stem cells, into vast numbers of progenitors comprising diverse cellular lineages which make up a complex immune system. Proper and balanced interactions between the cellular components are necessary for a healthy immune response. The different cellular lineages often respond in a different manner when cytokines are administered in conjunction with other agents.

Cell lineages especially important to the immune response include two classes of lymphocytes: B-cells, which can produce and secrete immunoglobulins (proteins with the capability of recognizing and binding to foreign matter to effect its removal), and T cells of various subsets that secrete cytokines and induce or suppress the B-cells and various other cells (including other T cells) making up the immune network. These lymphocytes interact with many other cell types.

Research to better understand and treat various immune disorders has been hampered by the general inability to maintain cells of the immune system in vitro. Immunologists have discovered that culturing many of these cells can be accomplished through the use of T cell and other cell supernatants, which contain various growth factors, including many of the cytokines.

Various growth and regulatory factors exist which modulate morphogenetic development. And many receptors for cytokines are also known. Often there are at least two critical subunits in the functional receptor. See, e.g., Gonda and D'Andrea (1997) Blood 89:355-369; Presky, et al. (1996) Proc. Natl Acad. Sci. USA 93:14002-14007; Drachman and Kaushansky (1995) Curr. Opin. Hematol. 2:22-28; Theze (1994) Eur. Cytokine Netw. 5:353-368,; and Lemmon and Schlessinger (1994) Trends Biochem. Sci. 19:459-463.

From the foregoing, it is evident that the discovery and development of new soluble proteins and their receptors, including ones similar to cytokines, should contribute to new therapies for a wide range of degenerative or abnormal conditions which directly or indirectly involve development, differentiation, or function, e.g., of the immune system and/or hematopoietic cells. In particular, the discovery and understanding of novel receptors for cytokine-like molecules which enhance or potentiate the beneficial activities of other cytokines would be highly advantageous. However, the lack of understanding of how the immune system is regulated or differentiates has blocked the ability to advantageously modulate the normal defensive mechanisms to biological challenges. Medical conditions characterized by abnormal or inappropriate regulation of the development or physiology of relevant cells thus remain unmanageable. The discovery and characterization of specific cytokines and their receptors will contribute to the development of therapies for a broad range of degenerative or other conditions which affect the immune system, hematopoietic cells, as well as other cell types. The present invention provides new receptors for ligands exhibiting similarity to cytokine like compositions and related compounds, and methods for their use.

### SUMMARY OF THE INVENTION

The present invention is based, in part, on the discovery that IL-17C (SEQ ID NO:24) is the ligand of DCRS9 (SEQ ID NO: 12).

The invention provides a method of modulating an activity of a cell comprising contacting the cell with an agonist or antagonist of DCRS9 (SEQ ID NOs:11 or 12) or of IL-7C (SEQ ID NOs:23 or 24), wherein the cell modulates psoriasis; inflammatory bowel disorder (IBD); interstitial lung disorder; asthma or allergy; or atherosclerosis.

Also provided is the above method wherein the cell is a monocyte or a macrophage; a dendritic cell; an epithelial cell; an endothelial cell; or a keratinocyte; or wherein the activity is cytokine expression. Moreover, the invention provides the above method, wherein the agonist or antagonist specifically binds to a polypeptide or nucleic acid of DCRS9 (SEQ ID NOs:11 or 12); or IL-17C (SEQ ID NOs:23 or 24); and the above method wherein the agonist or antagonist comprises a binding composition derived from an antigen binding site of an antibody that specifically binds to a polypeptide of DCRS9 (SEQ ID NO:12); or IL-17C (SEQ ID NO:24).

In another aspect, the invention provides the above method wherein the binding composition comprises a polyclonal antibody; a monoclonal antibody; a humanized antibody; an Fab, Fv, or F(ab′)₂ fragment; or a peptide mimetic of an antibody; as well as the above method wherein the agonist or antagonist comprises a soluble receptor derived from DCRS9 (SEQ ID NO:12). In yet another aspect, the invention provides the above method wherein the agonist or antagonist comprises an anti-sense nucleic acid; or an interference RNA nucleic acid.

In another aspect, the invention provides a method of treating a subject suffering from a disorder comprising psoriasis; inflammatory bowel disorder (IBD); interstitial lung disorder; asthma or allergy; or atherosclerosis; by administering an effective amount of an agonist or antagonist of DCRS9 (SEQ ID NOs:11 or 12) or IL-17C (SEQ ID NOs:23 or 24). Also provided is the above method of treating a subject, wherein the disorder is mediated by monocytes or macrophages; dendritic cells; epithelial cells; endothelial cells; or keratinocytes; as well as the above method wherein the IBD comprises Crohn's disease or ulcerative colitis, or wherein the interstial lung disorder comprises idiopathic pulmonary fibrosis, eosinophilic granuloma, or hypersensitivity pneumonitis.

In another embodiment, the invention provides the above method of treatment, wherein the agonist or antagonist specifically binds to a polypeptide or nucleic acid of DCRS9 (SEQ ID NOs:11 or 12); or IL-17C (SEQ ID NOs:23 or 24); as well as the above method wherein the agonist or antagonist is a binding composition derived from an antigen binding site of an antibody that specifically binds to a polypeptide of DCRS9 (SEQ ID NO:12); or IL-17C (SEQ ID NO:24). Moreover, the invention provides the above method wherein the binding composition comprises a polyclonal antibody; a monoclonal antibody; a humanized antibody; an Fab, Fv, or F(ab′)₂ fragment; or a peptide mimetic of an antibody; as well as the above method wherein the agonist or antagonist is a soluble receptor derived from DCRS9 (SEQ ID NO:24); or wherein the agonist or antagonist comprises an anti-sense nucleic acid; or an interference RNA nucleic acid.

Yet another embodiment of the invention is a method of diagnosing a disorder comprising contacting a sample from a test subject with a binding composition that specifically binds to a polypeptide or nucleic acid of DCRS9 (SEQ ID NOs:11 or 12); or IL-17C (SEQ ID NOs:23 or 24), where the disorder comprises psoriasis, IBD, interstial lung disorder, asthma or allergy, or atherosclerosis. Also provided is the above method of diagnosis, wherein the sample is derived from a tissue, cell, or biological fluid; as well as the above method further comprising contacting a sample from a normal subject or a control source with the binding composition; and comparing the binding to the test subject with the binding to the normal subject or control source.

The present invention is directed to novel receptors related to cytokine receptors, e.g., primate, cytokine receptor like molecular structures, designated DNAX Cytokine Receptor Subunits (DCRS), and their biological activities. In particular, it provides description of various subunits, designated DCRS6, DCRS7, DCRS8, DCRS9, and DCRS10. Primate, e.g, human, and rodent, e.g., mouse, embodiments of the various subunits are provided. It includes nucleic acids coding for the polypeptides themselves and methods for their production and use. The nucleic acids of the invention are characterized, in part, by their homology to cloned complementary DNA (cDNA) sequences enclosed herein.

The present invention provides a composition of matter selected from: a substantially pure or recombinant polypeptide comprising at least three distinct nonoverlapping segments of at least four amino acids identical to segments of SEQ ID NO: 2, 4, 6, 8, 16, 18; a substantially pure or recombinant polypeptide comprising at least three distinct nonoverlapping segments of at least four amino acids identical to segments of SEQ ID NO: 10; a substantially pure or recombinant polypeptide comprising at least two distinct nonoverlapping segments of at least five amino acids identical to segments of SEQ ID NO: 10; a natural sequence DCRS8 comprising mature SEQ ID NO: 10; a fusion polypeptide comprising DCRS8 sequence; a substantially pure or recombinant polypeptide comprising at least three distinct nonoverlapping segments of at least four amino acids identical to segments of SEQ ID NO: 12 or 14; a substantially pure or recombinant polypeptide comprising at least two distinct nonoverlapping segments of at least five amino acids identical to segments of SEQ ID NO: 12 or 14; a natural sequence DCRS9 comprising mature SEQ ID NO: 12 or 14; or a fusion polypeptide comprising DCRS9 sequence. Preferably, wherein the distinct nonoverlapping segments of identity include: one of at least eight amino acids; one of at least four amino acids and a second of at least five amino acids; at least three segments of at least four, five, and six amino acids, or one of at least twelve amino acids. In other embodiments, the: polypeptide: comprises a mature sequence of SEQ ID NO: 16 or 18; is an unglycosylated form of DCRS8 or DCRS9; is from a primate, such as a human; comprises at least seventeen amino acids of SEQ ID NO: 10 or 12; exhibits at least four nonoverlapping segments of at least seven amino acids of SEQ ID NO: 10 or 12; is a natural allelic variant of DCRS8 or DCRS9; has a length at least about 30 amino acids; exhibits at least two non-overlapping epitopes which are specific for a primate DCRS8 or DCRS9; is glycosylated; has a molecular weight of at least 30 kD with natural glycosylation; is a synthetic polypeptide; is attached to a solid substrate; is conjugated to another chemical moiety; is a 5-fold or less substitution from natural sequence; or is a deletion or insertion variant from a natural sequence.

The invention further embraces a composition comprising: a substantially pure DCRS8 or DCRS9 and another cytokine receptor family member; a sterile DCRS8 or DCRS9 polypeptide; the DCRS8 or DCRS9 polypeptide and a carrier, wherein the carrier is: an aqueous compound, including water, saline, and/or buffer; and/or formulated for oral, rectal, nasal, topical, or parenteral administration. Additional embodiments include a polypeptide comprising: mature protein sequence of SEQ ID NOs: 2, 4, 6, 8, 10, 12, 14, 16, and 18; a detection or purification tag, including a FLAG, His6, or Ig sequence; or sequence of another cytokine receptor protein. Kit embodiments include ones comprising a described polypeptide, and: a compartment comprising the protein or polypeptide; or instructions for use or disposal of reagents in the kit.

Binding compositions are provided, e.g., comprising an antigen binding site from an antibody, which specifically binds to a natural DCRS8 or DCRS9 polypeptide, wherein: the binding compound is in a container; the DCRS8 or DCRS9 polypeptide is from a human; the binding compound is an Fv, Fab, or Fab₂ fragment; the binding compound is conjugated to another chemical moiety; or the antibody: is raised against a peptide sequence of a mature polypeptide of SEQ ID NOS: 10, 12, or 14; is raised against a mature DCRS8 or DCRS9; is raised to a purified human DCRS8 or DCRS9; is immunoselected; is a polyclonal antibody; binds to a denatured DCRS8 or DCRS9; exhibits a Kd to antigen of at least 30 μM; is attached to a solid substrate, including a bead or plastic membrane; is in a sterile composition; or is detectably labeled, including a radioactive or fluorescent label. Kits include ones comprising such a binding compound, and: a compartment comprising the binding compound; or instructions for use or disposal of reagents in the kit.

The invention also provides methods of producing an antigen:antibody complex, comprising contacting under appropriate conditions a primate DCRS8 or DCRS9 polypeptide with a described antibody, thereby allowing the complex to form. Preferred methods include ones wherein: the complex is purified from other cytokine receptors; the complex is purified from other antibody; the contacting is with a sample comprising an interferon; the contacting allows quantitative detection of the antigen; the contacting is with a sample comprising the antibody; or the contacting allows quantitative detection of the antibody. Further compositions include those comprising: a sterile binding compound, as described, or the binding compound and a carrier, wherein the carrier is: an aqueous compound, including water, saline, and/or buffer; and/or formulated for oral, rectal, nasal, topical, or parenteral administration.

Nucleic acid compositions include an isolated or recombinant nucleic acid encoding a desribed polypeptide wherein the: DCRS8 or DCRS9 is from a human; or the nucleic acid: encodes an antigenic peptide sequence of SEQ ID NOS: 10, 12, or 14; encodes a plurality of antigenic peptide sequences of; exhibits identity over at least thirteen nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate; comprises a natural full length coding sequence; is a hybridization probe for a gene encoding the DCRS8 or DCRS9; or is a PCR primer, PCR product, or mutagenesis primer. Also provided are a cell or tissue comprising such a recombinant nucleic acid, e.g., where the cell is: a prokaryotic cell; a eukaryotic cell; a bacterial cell; a yeast cell; an insect cell; a mammalian cell; a mouse cell; a primate cell; or a human cell.

Kit embodiments include those comprising a described nucleic acid and: a compartment comprising the nucleic acid; a compartment further comprising a primate DCRS8 or DCRS9 polypeptide; or instructions for use or disposal of reagents in the kit.

Other nucleic acids provided include ones which: hybridize under wash conditions of 30 minutes at 30° C. and less than 2M salt to the coding portion of SEQ ID NO: 9 or 11; or exhibit identity over a stretch of at least about 30 nucleotides to a primate DCRS8 or DCRS9. Preferably, such will be nucleic acids where: the wash conditions are: at 45° C. and/or 500 mM salt; at 55° C. and/or 150 mM salt; or the stretch is at least 55 or 75 nucleotides.

Also provided are methods of modulating physiology or development of a cell or tissue culture cells comprising contacting the cell with an agonist or antagonist of a mammalian DCRS8 or DCRS9. Preferably, the cell is transformed with a nucleic acid encoding the DCRS8 or DCRS9 and another cytokine receptor subunit.

### DETAILED DESCRIPTION

I. General

The present invention provides the amino acid sequence and DNA sequence of mammalian, herein primate, cytokine receptor-like subunit molecules, these designated DNAX Cytokine Receptor Subunits 6 (DCRS6), 7 (DCRS7), 8 (DCRS8), 9 (DCRS9), and 10 (DCRS10) having particular defined properties, both structural and biological. Various cDNAs encoding these molecules were obtained from primate, e.g., human, and/or rodent, e.g., mouse, cDNA sequence libraries. Other primate or other mammalian counterparts would also be desired.

“Activation,” “stimulation,” and “treatment,” as it applies to cells or to receptors, may have the same meaning, e.g., activation, stimulation, or treatment of a cell or receptor with a ligand or suitable binding composition, unless indicated otherwise by the context or explicitly.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo. treatments, e.g., of a cell, by a reagent, diagnostic, binding composition, or by another cell. Treatment encompasses methods using a purified immune cell, e.g., in a mixed cell reactions or for administration to a research, animal, or human subject. The invention contemplates treatment with a cell, a purified cell, a stimulated cell, a cell population enriched in a particular cell, and a purified cell. Treatment further encompasses situations where an administered reagent or cell is modified by metabolism, degradation, or by conditions of storage.

“Contacting a cell” encompasses, e.g., a step, method, or procedure, where the addition or introduction of a compound or composition to a container, physiological compartment, fluid, or gel, that contains the cell, would be expected to result in the contact of the compound or composition to the cell. “Contacting a cell” also encompasses, e.g., a step, method, or procedure, where the added or introduced compound or composition is known to contact the cell.

“Control source,” as it applies to binding assays or diagnostic assays, refers to a source of, e.g., cells, tissue, or biological fluid. The control source can be derived from a normal, non-afflicted subject. The control source can also be derived from an afflicted subject, but from a normal, control, or non-afflicted part of the body of the test subject. Alternatively, the control source can be provided by a predetermined sample or by a predetermined quantity, value, or number. The term “test subject” encompasses any subject that is tested, including normal subjects, subjects suffering from a disorder or pathological condition, research subjects, experimental subjects, and veterinary subjects.

“Effective amount” or “therapeutically effective amount” means an amount sufficient to ameliorate a symptom or sign of a disorder or physiological condition or an amount sufficient to permit or facilitate a diagnosis of the disorder or physiological condition. An effective amount for a particular patient or veterinary subject may vary depending on factors such as the condition being treated, the overall health of the patient, the method route and dose of administration and the severity of side affects (see, e.g., U.S. Pat. No. 5,888,530 issued to Netti, et al.). An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

“Inhibitors” and “antagonists” or “activators” and “agonists” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity-or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis, see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126.

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

Where a cell “modulates” or “mediates,” e.g., a physiological state, a biological activity, pathological state, a disorder, or a disease, means that the state or disorder is partially or completely dependent on the presence of the cell or an activity of the cell. Where a cell “modulates” or “mediates” also encompasses situations where the cell's participation or activity is completely or partially redundant and duplicative of other mechanisms, that is, where dependency cannot be easily shown. A completely redundant or duplicative situation is one where a physiological or pathological state remains in effect, or would be expected to remain in effect, even when the cell in question is inactivated or removed. Where a cell “modulates” or “mediates” encompasses situations where direct cell contact mediates a physiological state, as well as situations were indirect cell contact, e.g., through a soluble signaling molecule, mediates a physiological state.

“Soluble receptor” refers to a receptor that is water-soluble and occurs, e.g., in an extracellular fluid, intracellular fluid, or weakly associated with a membrane. Soluble receptor further refers to a receptor that is engineered to be water soluble. Methods relating to soluble receptors are described (see, e.g., Monahan, et al. (1997) J. Immunol. 159:4024-4034; Moreland, et al. (1997) New Engl. J. Med. 337:141-147; Borish, et al. (1999) Am. J. Respir. Crit. Care Med. 160:1816-1823; Uchibayashi, et al. (1989) J. Immunol. 142:3901-3908; Jones, et al. (2002) Biochim. Biophys. Acta 1592:251-263; Prudhomme, et al. (2001) Expert Opinion Biol. Ther. 1:359-373; Fernandez-Botran (1999) Crit. Rev. Clin. Lab Sci. 36:165-224). Also contemplated are soluble receptors and cytokine derivatives comprising an Ig fusion protein (see, e.g., Harris, et al. (2002) J. Immunol. Methods 268:245-258; Corcoran, et al. (1998) Eur. Cytokine Netw. 9:255-262; Mackay, et al. (1997) Eur. J. Immunol. 27:2033-2042).

“Specificity of binding,” “selectivity of binding,” and the like, refer to a binding interaction between a predetermined ligand and a predetermined receptor that enables one to distinguish between the predetermined ligand and other ligands, or between the predetermined receptor and other receptors. “Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, binds to its antigen with an affinity that is at least two fold greater, preferably at least ten times greater, more preferably at least 20-times greater, and most preferably at least 100-times greater than the affinity to any other antigen. In a preferred embodiment the antibody will have an affinity that is greater than about 10⁹ liters/mol, see, e.g., Munsen, et al. (1980) Analyt. Biochem. 107:220-239).

“Symptom” of a disorder refers to a parameter that is specifically correlated with, or used as a measure of, a physiological state, a disorder, or pathological condition. The parameter may reflect, e.g., the clinical, histological, biochemical, or psychological features of the state, disorder, or condition. Symptoms of pulmonary disorders are assessed, e.g., by measuring airflow obstruction, airway hyperreactivity, inflammation, or structural changes in airways (see, e.g., Kumar (2001) Pharmacology and Therapeutics 91:93-104; Maddox and Schwartz (2002) Annu. Rev. Med. 53:477-498; Harris, et al. (2002) New Engl. J. Med. 347:1262-1268). Symtoms of IBD are assessed, e.g., by radiologic, endoscopic, histologic, and biochemical methods (see, e.g., Podolsky (2003) New Engl. J. Med. 347:417-429). Symptoms of psoriasis and inflammatory skin disorders are assessed, e.g., by erythema, induration, desquamation, percentage of body surface area affected, and by biochemical and histological parameters (see, e.g., Ellis, et al. (2001) New Engl. J. Med. 345:248-255; Bos and DeRie (1999) Immunol. Today 20:40-46).

Nucleotide (SEQ ID NO: 1) and corresponding amino acid sequence (SEQ ID NO: 2) of a primate, e.g., human, DCRS6 coding segment is shown. Rodent, e.g., mouse, counterpart sequences are provided, e.g., SEQ ID NO: 3 and SEQ ID NO:4.

Similarly, nucleotide (SEQ ID NO: 5) and corresponding amino acid sequence (SEQ ID NO: 6) of a primate, e.g., human, DCRS7 coding segment is shown. Rodent, e.g., mouse, counterpart sequences are provided, e.g., SEQ ID NO: 7 and SEQ ID NO: 8. Nucleotide (SEQ ID NO: 9) and corresponding amino acid sequence (SEQ ID NO: 10) of a primate, e.g., human, DCRS8 coding segment is shown.

Nucleotide (SEQ ID NO: 11) and corresponding amino acid sequence (SEQ ID NO: 12) of a primate, e.g., human, DCRS9 coding segment is shown. Rodent, e.g., mouse, counterpart sequences are provided, e.g., SEQ ID NO: 13 and SEQ ID NO: 14. Nucleotide (SEQ ID NO: 15) and corresponding amino acid sequence (SEQ ID NO: 16) of a primate, e.g., human, DCRS10 coding segment is shown. Rodent, e.g., mouse, counterpart sequences are provided, e.g., SEQ ID NO: 17 and SEQ ID NO: 18.

TABLE 1

Alignment of the cytoplasmic portions of various cytokine receptor
subunits are shown.

```
              The indicated segments are: amino acids 477-658 of SEQ ID NO: 8 (DCRS7_M)
amino acids 476-662 of SEQ ID NO: 6 (DCRS7_Hu); amino acids 401-565 of SEQ ID
NO: 16 (DCRS10_Hu); amino acids 178-341 of SEQ ID NO: 18 (DCRS10_Mu); amino
acids 455-620 of SEQ ID NO: 12 (DCRS9_Hu); amino acids 339-542 of SEQ ID NO: 1
(DCRS8_Hu); and amino acids 318-488 of SEQ ID NO: 2 (DCRS6_Hu) (Table 1).
              The IL-17R_Hu (SEQ ID NO: 19) is GenBank AAB99730.1(U58917),
gi|7657230; the IL-17R_Mu (SEQ ID NO: 20) is GenBank AAC52357.1(U31993),
gi|6680411; the IL-17R_Ce (SEQ ID NO: 21) is GenBank AAA811100.1(U39997),
gi|1353171; and the DCRS6_Ce (SEQ ID NO: 22) is EMBCAA90543.1(Z50177),
gi|7503597.
```

| | |
|---|---|
| DCRS7_Mu | RTALLLHSADG-AGYERLVGALASALSQMP---LRVAVDLWSRRE-LSAHGALAWFHHQR |
| DCRS7_Hu | RAALLLYSADD-SGFERLVGALASALCQLP---LRVAVDLWSRRE-LSAQGPVAWFHAQR |
| IL-17R_Hu | RKVWIIYSADH-PLYVDVVLKFAQFLLTACG--TEVALDLLEEQA-ISEAGVMTWVGRQK |
| IL-17R_Mu | RKVWIVYSADH-PLYVEVVLKFAQFLITACG--TEVALDLLEEQV-ISEVGVMTWVSRQK |
| DCRS10 | RKVFITYSMD----TAMEVVKFVNFLLVNG---FQTAIDIFEDR--IRGIDIIKWMERYL |
| DCRS10_Mu | RKVFITYSMD----TAMEVVKFVNFLLVNG---FQTAIDIFEDR--IRGIDIIKWMERYL |
| DCRS9_Hu | RPVLLLHAADS-EAQRRLVGALAELLRAALGGGRDVIVDLWEGRH-VARVGPLPWLWAAR |
| DCRS8_Hu | PKVFLCYSSKDGQNHMNVVQCFAYFLQDFCG--CEVALDLWEDFS-LCREGQREWVIQKI |
| IL-17R_Ce | VKVMIVYADDN-DLHTDCVKKLVENLRNCAS--CDPVFDLEKLI--TAEIVPSRWLVDQI |
| DCRS6_Hu | IKVLVVYPSEI--CFHHTICYFTEFLQNHCR--SEVILEKWQKKK-IAEMGPVQWLATQK |
| DCRS6_Ce | FKVMLVCPEVS-GRDEDFMMRIADALKKSN---NKVVCDRWFEDSKNAEENMLHWVYEQT |
| | . : . : :. * : *. |
| DCRS7_Mu | RRILQEGGVVILLFSPAAVAQCQ---QWLQLQTVEP---GP---HDALAAWLSCVLPDFL |
| DCRS7_Hu | RQTLQEGGVVVLLFSPGAVALCS---EWLQDGVSGPGAHGP---HDAFRASLSCVLPDFL |
| IL-17R_Hu | QEMVESNSKIIVLCSRGTRAKWQALLGRGAP-VRLRCDHGKPV-GDLFTAAMNMILPDFK |
| IL-17R_Mu | QEMVESNSKIIILCSRGTQAKWKAILGWAEPAVQLRCDHWKPA-GDLFTAANNMILPDFK |
| DCRS10 | R---DKTVMIIVAISPKYKQDVE----GAESQLDED-EHGL---HTKYIHRM-MQIEFIK |
| DCRS10_Mu | R---DKTVMIIVAISPKYKQDVE----GAESQLDED-EHGL---HTKYIHRM-MQIEFIS |
| DCRS9_Hu | TRVAREQGTVLLLWSGADLRPVS----GPDP-RAAP-----------LLA----LLHAAP |
| DCRS8_Hu | H----ESQFIIVVCSKGMKYFVD---KKNYKHKGGGRGSGK---GELFLVAVSAIAEKLR |
| IL-17R_Ce | S----SLKKFIIVVSDCAEKILD----TEASETHQLVQARP--FADLFGPAMEMIIRDAT |
| DCRS6_Hu | K----AADKVVFLLSNDVNSVCD----GTCGKSEGSPSENS---QDLFPLAFNLFCSDLR |
| DCRS6_Ce | K----IAEKIIVFHSAYYHPRCG---IYDVINNFFPCTDPR-----LAHIALT---PEAQ |
| | .:. * |
| DCRS7_Mu | QGRATGR-----YVGVYFDGLLHPDSVPSPFRVAPLFSLP-SQLPAFLDALQ--GGCSTS |
| DCRS7_Hu | QGRAPGS-----YVGACFDRLLHPDAVPALFRTVPVFTLP-SQLPDFLGALQ--QPPAPR |
| IL-17R_Hu | RPACFGT-----YVVCYFSEVSCDGDVPDLFGAAPRYPLM-DRFEEVYFRIQ--DLEMFQ |
| IL-17R_Mu | RPACFGT-----YVVCYFSGICSERDVPDLFNITSRYPLM-DRFEEVYFRIQ--DLEMFE |
| DCRS10 | QGSMNFR-----FIPVLFPNAK-KEHVPTWLQNTHVYSWP-KNKKNILLRLL-REEEYVA |
| DCRS10_Mu | QGSMNFR-----FIPVLFPNAK-KEHVPTWLQNTHVYSWP-KNKKNILLRLL-REEEYVA |
| DCRS9_Hu | RPL---------LLLAYFSRLCAKGDIPPPLRALPRYRLL-RDLPRLLRALD--ARPFAE |
| DCRS8_Hu | QAKQSSSAALSKFIAVYFDYSC-EGDVPGILDLSTKYRLM-DNLPQLCSHLHSRDHGLQE |
| IL-17R_Ce | HNFPEAR---KKYAVVRFNYSP---HVPPNLAILNLPTFIPEQFAQLTAFLHN-VEHTER |
| DCRS6_Hu | SQIHLHK-----YVVVYFREID-TKDDYNALSVCPKYHLM-KDATAFCAELL---HVKQQ |
| DCRS6_Ce | RSVPKEV----EYVLPRDQKLL--EDAFDITIADPLVIDIPIEDVAIPENVP--IHHESC |
| | . : |
| DCRS7_Mu | AGRPADRVER-----VT----QALRSALDSCTS----------- |
| DCRS7_Hu | SGRLQERAEQ-----VS----RALQPALDSYFHPP--------- |
| IL-17R_Hu | PGRMHRVGELSGDNYLRS---PGGRQLRAALDRFRDWQVRCPDW |
| IL-17R_Mu | PGRMHHVRELTGDNYLQS---PSGRQLKEAVLRFQEWQTQCPDW |
| DCRS10 | P----PRGPL-----------PTLQVVPL--------------- |
| DCRS10_Mu | P----PRGPL-----------PTLQVVPL--------------- |
| DCRS9_Hu | ATSWGRLGAR-----------QRRQSRLELCSR----------- |
| DCRS8_Hu | PGQHTRQGSR-----RNYFRSKSGRSLYVAICNMHQFIDEEPDW |
| IL-17R_Ce | ANVTQNISEA------Q------IHEWNLCASRMMSFFVRNPNW |
| DCRS6_Hu | VS----AGKR-------------SQACHDGCCSL---------- |
| DCRS6_Ce | DSIDSRNNSK-------------THSTDSGVSSLSS----NS- |

Of particular interest are motifs or features corresponding, in primate DCRS8 to: R/K at 339/340; D/E at 348/349; alpha helical regions from H353-Q365, C370-S381, E389-H396, K410-D414, and D485-H495; beta sheet regions correspond to F400-V404 and F458-Y462; E at 431; E/D at 442/443; Y/F at 458; D/E at 468-470; Y/F at 481; and Q/R/F at 523 (Table 1).

Table 1 shows comparison of the available sequences of primate, rodent, and various other receptors. Various conserved residues are aligned and indicated. The structurally homologous cytoplasmic domains most likely signal through pathways like IL-17, e.g., through NFkB. Similar to IL-1 signaling, it is likely that these receptors are involved in innate immunity and/or development.

As used herein, the term DCRS shall be used to describe a protein comprising amino acid sequences shown in SEQ ID NOS: 2, 4, 6, 8, 10, 12, 14, 16, and 18 , respectively. In many cases, a substantial fragment thereof will be functionally or structurally equivalent, including, e.g., an extracellular or intracellular domain. The invention also includes a protein variation of the respective DCRS allele whose sequence is provided, e.g., a mutein or soluble extracellular construct. Typically, such agonists or antagonists will exhibit less than about 10% sequence differences, and thus will often have between 1 and 11 substitutions, e.g., 2-, 3-, 5-, 7-fold, and others. It also encompasses allelic and other variants, e.g., natural polymorphic, of the protein described. Typically, it will bind to its corresponding biological ligand, perhaps in a dimerized state with an alpha receptor subunit, with high affinity, e.g., at least about 100 nM, usually better than about 30 nM, preferably better than about 10 nM, and more preferably at better than about 3 nM. The term shall also be used herein to refer to related naturally occurring forms, e.g., alleles, polymorphic variants, and metabolic variants of the mammalian protein. Preferred forms of the receptor complexes will bind the appropriate ligand with an affinity and selectivity appropriate for a ligand-receptor interaction.

This invention also encompasses combinations of proteins or peptides having substantial amino acid sequence identity with an amino acid sequence in SEQ ID NOS: 2, 4, 6, 8, 10, 12, 14, 16, or 18. It will include sequence variants with relatively few residue substitutions, e.g., preferably less than about 3-5.

A substantial polypeptide “fragment”, or “segment”, is a stretch of amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in particularly preferred embodiments, at least about 30 or more amino acids. This includes, e.g., 40, 50, 60, 70, 85, 100, 115, 130, 150, and other lengths. Sequences of segments of different proteins can be compared to one another over appropriate length stretches, typically between conserved motifs. In many situations, fragments may exhibit functional properties of the intact subunits, e.g., the extracellular domain of the transmembrane receptor may retain the ligand binding features, and may be used to prepare a soluble receptor-like complex.

Amino acid sequence homology, or sequence identity, is determined by optimizing residue matches. In some comparisons, gaps may be introduces, as required. See, e.g., Needleham, et al. (1970) J. Mol. Biol. 48:443-453; Sankoff, et al., (1983) chapter one in Time Warps, String Edits, and Macromolecules: The Theory and Practice of Sequence Comparison, Addison-Wesley, Reading, Mass.; and software packages from IntelliGenetics, Mountain View, Calif.; and the University of Wisconsin Genetics Computer Group (GCG), Madison, Wis. This changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences are intended to include natural allelic and interspecies variations in the cytokine sequence. Typical homologous proteins or peptides will have from 50-100% homology (if gaps can be introduced), to 60-100% homology (if conservative substitutions are included) with an amino acid sequence segment of, e.g., SEQ ID NO: 10, 12, or 14. Homology measures will be at least about 70%, generally at least 76%, more generally at least 81%, often at least 85%, more often at least 88%, typically at least 90%, more typically at least 92%, usually at least 94%, more usually at least 95%, preferably at least 96%, and more preferably at least 97%, and in particularly preferred embodiments, at least 98% or more. The degree of homology will vary with the length of the compared segments. Homologous proteins or peptides, such as the allelic variants, will share most biological activities with the embodiments described in SEQ ID NOS: 2, 4, 6, 8, 10, 12, 14, 16, and 18.

As used herein, the term “biological activity” is used to describe, without limitation, effects on inflammatory responses, innate immunity, and/or morphogenic development by cytokine-like ligands. For example? these receptors should mediate phosphatase or phosphorylase activities, which activities are easily measured by standard procedures. See, e.g., Hardie, et al. (eds.) (1995) The Protein Kinase FactBook, vols. I and II, Academic Press, San Diego, Calif.; Hanks, et al. (1991) Meth. Enzymol. 200:38-62; Hunter, et al. (1992) Cell 70:375-388; Lewin (1990) Cell 61:743-752; Pines, et al. (1991) Cold Spring Harbor Symp. Quant. Biol. 56:449-463; and Parker, et al. (1993) Nature 363:736-738. The receptors, or portions thereof, may be useful as phosphate labeling enzymes to label general or specific substrates. The subunits may also be functional immunogens to elicit recognizing antibodies, or antigens capable of binding antibodies.

The terms ligand, agonist, antagonist, and analog of, e.g., an IL-17C (SEQ ID NOs:23, 24) or DCRS9 (SEQ ID NOs:11, 12) include molecules that modulate the characteristic cellular responses to cytokine ligand proteins, as well as molecules possessing the more standard structural binding competition features of ligand-receptor interactions, e.g., where the receptor is a natural receptor or an antibody. The cellular responses likely are typically mediated through receptor tyrosine kinase pathways.

Also, a ligand is a molecule which serves either as a natural ligand to which said receptor, or an analog thereof, binds, or a molecule which is a functional analog of the natural ligand. The functional analog may be a ligand with structural modifications, or may be a wholly unrelated molecule which has a molecular shape which interacts with the appropriate ligand binding determinants. The ligands may serve as agonists or antagonists, see, e.g., Goodman, et al. (eds.) (1990) Goodman & Gilman's: The Pharmacological Bases of Therapeutics, Pergamon Press, New York.

Rational drug design may also be based upon structural studies of the molecular shapes of a receptor or antibody and other effectors or ligands. See, e.g., Herz, et al. (1997) J. Recept. Signal Transduct. Res. 17:671-776; and Chaiken, et al. (1996) Trends Biotechnol. 14:369-375. Effectors may be other proteins which mediate other functions in response to ligand binding, or other proteins which normally interact with the receptor. One means for determining which sites interact with specific other proteins is a physical structure determination, e.g., x-ray crystallography or 2 dimensional NMR techniques. These will provide guidance as to which amino acid residues form molecular contact regions. For a detailed description of protein structural determination, see, e.g., Blundell and Johnson (1976) Protein Crystallography, Academic Press, New York.

II. Activities

The cytokine receptor-like proteins will have a number of different biological activities, e.g., modulating cell proliferation, or in phosphate metabolism, being added to or removed from specific substrates, typically proteins. Such will generally result in modulation of an inflammatory function, other innate immunity response, or a morphological effect. The subunit will probably have a specific low affinity binding to the ligand.

The DCRS8 and DCRS9 have characteristic motifs of receptors signaling through the JAK pathway. See, e.g., Ihle, et al. (1997) Stem Cells 15 (suppl. 1):105-111; Silvennoinen, et al. (1997) APMIS 105:497-509; Levy (1997) Cytokine Growth Factor Review 8:81-90; Winston and Hunter (1996) Current Biol. 6:668-671; Barrett (1996) Baillieres Clin. Gastroenterol. 10: 1-15; and Briscoe, et al. (1996) Philos. Trans. R. Soc. Lond. B. Biol. Sci. 351:167-171.

The biological activities of the cytokine receptor subunits will be related to addition or removal of phosphate moieties to substrates, typically in a specific manner, but occasionally in a non specific manner. Substrates may be identified, or conditions for enzymatic activity may be assayed by standard methods, e.g., as described in Hardie, et al. (eds. 1995) The Protein Kinase FactBook vols. I and II, Academic Press, San Diego, Calif.; Hanks, et al. (1991) Meth. Enzymol. 200:38-62; Hunter, et al. (1992) Cell 70:375-388; Lewin (1990) Cell 61:743-752; Pines, et al. (1991) Cold Spring Harbor Symp. Quant. Biol. 56:449-463; and Parker, et al. (1993) Nature 363:736-738.

The receptor subunits may combine to form functional complexes, e.g., which may be useful for binding ligand or preparing antibodies. These will have substantial diagnostic uses, including detection or quantitation.

III. Nucleic Acids

This invention contemplates use of isolated nucleic acid or fragments, e.g., which encode these or closely related proteins, or fragments thereof, e.g., to encode a corresponding polypeptide, preferably one which is biologically active. In addition, this invention covers isolated or recombinant DNAs which encode combinations of such proteins or polypeptides having characteristic sequences, e.g., of the DCRSs. Typically, the nucleic acid is capable of hybridizing, under appropriate conditions, with a nucleic acid sequence segment shown in SEQ ID NOS: 1, 3, 5, 7, 9, 11, 13, 15, and 17, but preferably not with a corresponding segment of other receptors described in Table 1. Said biologically active protein or polypeptide can be a full length protein, or fragment, and will typically have a segment of amino acid sequence highly homologous, e.g., exhibiting significant stretches of identity, to one shown in SEQ ID NOS: 1, 3, 5, 7, 9, 11, 13, 15, and 17. Further, this invention covers the use of isolated or recombinant nucleic acid, or fragments thereof, which encode proteins having fragments which are equivalent to the IL-17C or DCRS9 proteins. The isolated nucleic acids can have the respective regulatory sequences in the 5′ and 3′ flanks, e.g., promoters, enhancers, poly-A addition signals, and others from the natural gene. Combinations, as described, are also provided.

An “isolated” nucleic acid is a nucleic acid, e.g., an RNA, DNA, or a mixed polymer, which is substantially pure, e.g., separated from other components which naturally accompany a native sequence, such as ribosomes, polymerases, and flanking genomic sequences from the originating species. The term embraces a nucleic acid sequence which has been removed from its naturally occurring environment, and includes recombinant or cloned DNA isolates, which are thereby distinguishable from naturally occurring compositions, and chemically synthesized analogs or analogs biologically synthesized by heterologous systems. A substantially pure molecule includes isolated forms of the molecule, either completely or substantially pure.

An isolated nucleic acid will generally be a homogeneous composition of molecules, but will, in some embodiments, contain heterogeneity, preferably minor. This heterogeneity is typically found at the polymer ends or portions not critical to a desired biological function or activity.

A “recombinant” nucleic acid is typically defined either by its method of production or its structure. In reference to its method of production, e.g., a product made by a process, the process is use of recombinant nucleic acid techniques, e.g., involving human intervention in the nucleotide sequence. Typically this intervention involves in vitro manipulation, although under certain circumstances it may involve more classical animal breeding techniques. Alternatively, it can be a nucleic acid made by generating a sequence comprising fusion of two fragments which are not naturally contiguous to each other, but is meant to exclude products of nature, e.g., naturally occurring mutants as found in their natural state. Thus, for example, products made by transforming cells with an unnaturally occurring vector is encompassed, as are nucleic acids comprising sequence derived using any synthetic oligonucleotide process. Such a process is often done to replace a codon with a redundant codon encoding the same or a conservative amino acid, while typically introducing or removing a restriction enzyme sequence recognition site. Alternatively, the process is performed to join together nucleic acid segments of desired functions to generate a single genetic entity comprising a desired combination of functions not found in the commonly available natural forms, e.g., encoding a fusion protein. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide. This will include a dimeric repeat. Specifically included are synthetic nucleic acids which, by genetic code redundancy, encode equivalent polypeptides to fragments of DCRSs and fusions of sequences from various different related molecules, e.g., other cytokine receptor family members.

A “fragment” in a nucleic acid context is a contiguous segment of at least about 17 nucleotides, generally at least 21 nucleotides, more generally at least 25 nucleotides, ordinarily at least 30 nucleotides, more ordinarily at least 35 nucleotides, often at least 39 nucleotides, more often at least 45 nucleotides, typically at least 50 nucleotides, more typically at least 55 nucleotides, usually at least 60 nucleotides, more usually at least 66 nucleotides, preferably at least 72 nucleotides, more preferably at least 79 nucleotides, and in particularly preferred embodiments will be at least 85 or more nucleotides. Typically, fragments of different genetic sequences can be compared to one another over appropriate length stretches, particularly defined segments such as the domains described below.

A nucleic acid which codes for IL-17C or DCRS9 will be particularly useful to identify genes, mRNA, and cDNA species which code for itself or closely related proteins, as well as DNAs which code for polymorphic, allelic, or other genetic variants, e.g., from different individuals or related species. Preferred probes for such screens are those regions of the interleukin which are conserved between different polymorphic variants or which contain nucleotides which lack specificity, and will preferably be full length or nearly so. In other situations, polymorphic variant specific sequences will be more useful.

This invention further covers recombinant nucleic acid molecules and fragments having a nucleic acid sequence identical to or highly homologous to the isolated DNA set forth herein. In particular, the sequences will often be operably linked to DNA segments which control transcription, translation, and DNA replication. These additional segments typically assist in expression of the desired nucleic acid segment.

Homologous, or highly identical, nucleic acid sequences, when compared to one another, e.g., IL-17C or DCRS9 sequences, exhibit significant similarity. The standards for homology in nucleic acids are either measures for homology generally used in the art by sequence comparison or based upon hybridization conditions. Comparative hybridization conditions are described in greater detail below.

Substantial identity in the nucleic acid sequence comparison context means either that the segments, or their complementary strands, when compared, are identical when optimally aligned, with appropriate nucleotide insertions or deletions, in at least about 60% of the nucleotides, generally at least 66%, ordinarily at least 71%, often at least 76%, more often at least 80%, usually at least 84%, more usually at least 88%, typically at least 91%, more typically at least about 93%, preferably at least about 95%, more preferably at least about 96 to 98% or more, and in particular embodiments, as high at about 99% or more of the nucleotides, including, e.g., segments encoding structural domains such as the segments described below. Alternatively, substantial identity will exist when the segments will hybridize under selective hybridization conditions, to a strand or its complement, typically using a sequence derived from SEQ ID NOS: 1, 3, 5, 7, 9, 11, 13, 15, and 17. Typically, selective hybridization will occur when there is at least about 55% homology over a stretch of at least about 14 nucleotides, more typically at least about 65%, preferably at least about.75%, and more preferably at least about 90%. See, Kanehisa (1984) Nucl. Acids Res. 12:203-213. The length of homology comparison, as described, may be over longer stretches, and in certain embodiments will be over a stretch of at least about 17 nucleotides, generally at least about 20 nucleotides, ordinarily at least about 24 nucleotides, usually at least about 28 nucleotides, typically at least about 32 nucleotides, more typically at least about 40 nucleotides, preferably at least about 50 nucleotides, and more preferably at least about 75 to 100 or more nucleotides. This includes, e.g., 125, 150, 175, 200, 225, 246, 273, and other lengths.

Stringent conditions, in referring to homology in the hybridization context, will be stringent combined conditions of salt, temperature, organic solvents, and other parameters typically controlled in hybridization reactions. Stringent temperature conditions will usually include temperatures in excess of about 30° C., more usually in excess of about 37° C., typically in excess of about 45° C., more typically in excess of about 55° C., preferably in excess of about 65° C., and more preferably in excess of about 70° C. Stringent salt conditions will ordinarily be less than about 500 mM, usually less than about 400 mM, more usually less than about 300 mM, typically less than about 200 mM, preferably less than about 100 mM, and more preferably less than about 80 mM, even down to less than about 20 mM. However, the combination of parameters is much more important than the measure of any single parameter. See, e.g., Wetmur and Davidson (1968) J. Mol. Biol. 31:349-370.

The isolated DNA can be readily modified by nucleotide substitutions, nucleotide deletions, nucleotide insertions, and inversions of nucleotide stretches. These modifications result in novel DNA sequences which encode this protein or its derivatives. These modified sequences can be used to produce mutant proteins (muteins) or to enhance the expression of variant species. Enhanced expression may involve gene amplification, increased transcription, increased translation, and other mechanisms. Such mutant IL-17C or DCRS9-like derivatives include predetermined or site-specific mutations of the protein or its fragments, including silent mutations using genetic code degeneracy. “Mutant L-17C” or “mutant DCRS9” as used herein encompasses a polypeptide otherwise falling within the homology definition of the IL-17C or DCRS9 as set forth above, but having an amino acid sequence which differs from that of other cytokine or cytokine receptor-like proteins as found in nature, whether by way of deletion, substitution, or insertion.

Although site specific mutation sites are predetermined, mutants need not be site specific. Mammalian IL-17C (SEQ ID NOs:23, 24) or DCRS9 (SEQ ID NOs:11,12) mutagenesis can be achieved by making amino acid insertions or deletions in the gene, coupled with expression. Substitutions, deletions, insertions, or many combinations may be generated to arrive at a final construct. Insertions include amino- or carboxy-terminal fusions. Random mutagenesis can be conducted at a target codon and the expressed mammalian DCRS mutants can then be screened for the desired activity, providing some aspect of a structure-activity relationship. Methods for making substitution mutations at predetermined sites in DNA having a known sequence are well known in the art, e.g., by M13 primer mutagenesis. See also Sambrook, et al. (1989), supra, and Ausubel, et al. (1987 and periodic Supplements), supra.

The mutations in the DNA normally should not place coding sequences out of reading frames and preferably will not create complementary regions that could hybridize to produce secondary mRNA structure such as loops or hairpins.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence.

Polymerase chain reaction (PCR) techniques can often be applied in mutagenesis. Alternatively, mutagenesis primers are commonly used methods for generating defined mutations at predetermined sites. See, e.g., Innis, et al. (eds.) (1990) PCR Protocols: A Guide to Methods and Applications, Academic Press, San Diego, Calif.; and Dieffenbach and Dveksler (eds). (1995) PCR Primer: A Laboratory Manual, Cold Spring Harbor Press, CSH, N.Y.

Certain embodiments of the invention are directed to combination compositions comprising the receptor or ligand sequences described. In other embodiments, functional portions of the sequences may be joined to encode fusion proteins. In other forms, variants of the described sequences may be substituted.

IV. Proteins, Peptides

As described above, the present invention encompasses primate DCRS6-10, e.g., whose sequences are disclosed in SEQ ID NOS: 2, 4, 6, 8, 10, 12, 14, 16, and 18, as well as IL-17C. Allelic and other variants are also contemplated, including, e.g., fusion proteins combining portions of such sequences with others, including, e.g., epitope tags and functional domains.

The present invention also provides recombinant proteins, e.g., heterologous fusion proteins using segments from these primate or rodent proteins. A heterologous fusion protein is a fusion of proteins or segments which are naturally not normally fused in the same manner. Thus, the fusion product of, e.g., an IL-17C or DCRS9 with another cytokine receptor is a continuous protein molecule having sequences fused in a typical peptide linkage, typically made as a single translation product and exhibiting properties, e.g., sequence or antigenicity, derived from each source peptide. A similar concept applies to heterologous nucleic acid sequences: Combinations of various designated proteins into complexes are also provided.

In addition, new constructs may be made from combining similar functional or structural domains from other related proteins, e.g., cytokine receptors or Toll-like receptors, including species variants. For example, ligand-binding or other segments may be “swapped” between different new fusion polypeptides or fragments. See, e.g., Cunningham, et al. (1989) Science 243:1330-1336; and O'Dowd, et al. (1988) J. Biol. Chem. 263:15985-15992. Thus, new chimeric polypeptides exhibiting new combinations of specificities will result from the functional linkage of receptor-binding specificities. For example, the ligand binding domains from other related receptor molecules may be added or substituted for other domains of this or related proteins. The resulting protein will often have hybrid function and properties. For example, a fusion protein may include a targeting domain which may serve to provide sequestering of the fusion protein to a particular subcellular organelle.

Candidate fusion partners and sequences can be selected from various sequence data bases, e.g., GenBank, c/o IntelliGenetics, Mountain View, Calif.; and BCG, University of Wisconsin Biotechnology Computing Group, Madison, Wis. In particular, combinations of polypeptide sequences provided in SEQ ID NOS: 2, 4, 6, 8, 10, 12, 14, 16, and 18 are particularly preferred. Variant forms of the proteins may be substituted in the described combinations.

The present invention particularly provides muteins which bind cytokine-like ligands, and/or which are affected in signal transduction. Structural alignment of human DCRSs with other members of the cytokine receptor family show conserved features/residues. Alignment of the human DCRS8 sequence with other members of the cytokine receptor family indicates various structural and functionally shared features. See also, Bazan, et al. (1996) Nature 379:591; Lodi, et al. (1994) Science 263:1762-1766; Sayle and Milner-White (1995) TIBS 20:374-376; and Gronenberg, et al. (1991) Protein Engineering 4:263-269.

Substitutions with either mouse sequences or human sequences are particularly preferred. Conversely, conservative substitutions away from the ligand binding interaction regions will probably preserve most signaling activities; and conservative substitutions away from the intracellular domains will probably preserve most ligand binding properties.

“Derivatives” of the primate IL-17C (SEQ ID NOs:23, 24) or DCRS9 (SEQ ID NOs:11, 12) include amino acid sequence mutants, glycosylation variants, metabolic derivatives and covalent or aggregative conjugates with other chemical moieties. Covalent derivatives can be prepared by linkage of functionalities to groups which are found in the IL-17C or DCRS9 amino acid side chains or at the N- or C-termini, e.g., by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties, including C3 to C 18 normal alkyl, thereby forming alkanoyl aroyl species.

In particular, glycosylation alterations are included, e.g., made by modifying the glycosylation patterns of a polypeptide during its synthesis and processing, or in further processing steps. Particularly preferred means for accomplishing this are by exposing the polypeptide to glycosylating enzymes derived from cells which normally provide such processing, e.g., mammalian glycosylation enzymes. Deglycosylation enzymes are also contemplated. Also embraced are versions of the same primary amino acid sequence which have other minor modifications, including phosphorylated amino acid residues, e.g., phosphotyrosine, phosphoserine, or phosphothreonine.

A major group of derivatives are covalent conjugates of the receptors or fragments thereof with other proteins of polypeptides. These derivatives can be synthesized in recombinant culture such as N- or C-terminal fusions or by the use of agents known in the art for their usefulness in cross-linking proteins through reactive side groups. Preferred derivatization sites with cross-linking agents are at free amino groups, carbohydrate moieties, and cysteine residues.

Fusion polypeptides between the receptors and other homologous or heterologous proteins are also provided. Homologous polypeptides may be fusions between different receptors, resulting in, for instance, a hybrid protein exhibiting binding specificity for multiple different cytokine ligands, or a receptor which may have broadened or weakened specificity of substrate effect. Likewise, heterologous fusions may be constructed which would exhibit a combination of properties or activities of the derivative proteins. Typical examples are fusions of a reporter polypeptide, e.g., luciferase, with a segment or domain of a receptor, e.g., a ligand-binding segment, so that the presence or location of a desired ligand may be easily determined. See, e.g., Dull, et al., U.S. Pat. No. 4,859,609. Other gene fusion partners include glutathione-S-transferase (GST), bacterial β-galactosidase, trpE, Protein A, β-lactamase, alpha amylase, alcohol dehydrogenase, and yeast alpha mating factor. See, e.g., Godowski, et al. (1988) Science 241:812-816. Labeled proteins will often be substituted in the described combinations of proteins.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence.

Such polypeptides may also have amino acid residues which have been chemically modified by phosphorylation, sulfonation, biotinylation, or the addition or removal of other moieties, particularly those which have molecular shapes similar to phosphate groups. In some embodiments, the modifications will be useful labeling reagents, or serve as purification targets, e.g., affinity ligands.

Fusion proteins will typically be made by either recombinant nucleic acid methods or by synthetic polypeptide methods. Techniques for nucleic acid manipulation and expression are described generally, for example, in Sambrook, et al. (1989) Molecular Cloning: A Laboratory Manual (2nd ed.), Vols. 1-3, Cold Spring Harbor Laboratory, and Ausubel, et al. (eds.) (1987 and periodic supplements) Current Protocols in Molecular Biology, Greene/Wiley, New York. Techniques for synthesis of polypeptides are described, for example, in Merrifield (1963) J. Amer. Chem. Soc. 85:2149-2156; Merrifield (1986) Science 232: 341-347; and Atherton, et al. (1989) Solid Phase Peptide Synthesis: A Practical Approach, IRL Press, Oxford. See also Dawson, et al. (1994) Science 266:776-779 for methods to make larger polypeptides.

This invention also contemplates the use of derivatives of a IL-17C or DCRS9 other than variations in amino acid sequence or glycosylation. Such derivatives may involve covalent or aggregative association with chemical moieties. These derivatives generally fall into three classes: (1) salts, (2) side chain and terminal residue covalent modifications, and (3) adsorption complexes, for example with cell membranes. Such covalent or aggregative derivatives are useful as immunogens, as reagents in immunoassays, or in purification methods such as for affinity purification of a receptor or other binding molecule, e.g., an antibody. For example, a cytokine ligand can be immobilized by covalent bonding to a solid support such as cyanogen bromide-activated Sepharose®), by methods which are well known in the art, or adsorbed onto polyolefin surfaces, with or without glutaraldehyde cross-linking, for use in the assay or purification of a cytokine receptor, antibodies, or other similar molecules. The ligand can also be labeled with a detectable group, for example radioiodinated by the chloramine T procedure, covalently bound to rare earth chelates, or conjugated to another fluorescent moiety for use in diagnostic assays.

A combination, e.g., including an IL-17C or DCRS9, of this invention can be used as an immunogen for the production of antisera or antibodies specific, e.g., capable of distinguishing between other cytokine receptor family members, for the combinations described. The complexes can be used to screen monoclonal antibodies or antigen-binding fragments prepared by immunization with various forms of impure preparations containing the protein. In particular, the term “antibodies” also encompasses antigen binding fragments of natural antibodies, e.g., Fab, Fab₂, Fv, etc. The purified IL-17C or DCRS9 can also be used as a reagent to detect antibodies generated in response to the presence of elevated levels of expression, or immunological disorders which lead to antibody production to the endogenous receptor. Additionally, IL-17C or DCRS9 fragments may also serve as immunogens to produce the antibodies of the present invention, as described immediately below. For example, this invention contemplates antibodies having binding affinity to or being raised against the amino acid sequences shown in SEQ ID NOS: 2, 4, 6, 8, 10, 12, 14, 16, and 18, fragments thereof, or various homologous peptides. In particular, this invention contemplates antibodies having binding affinity to, or having been raised against, specific fragments which are predicted to be, or actually are, exposed at the exterior protein surface of the native IL-17C or DCRS9. Complexes of combinations of proteins will also be useful, and antibody preparations thereto can be made.

The blocking of physiological response to the receptor ligands may result from the inhibition of binding of the ligand to the receptor, likely through competitive inhibition. Thus, in vitro assays of the present invention will often use antibodies or antigen binding segments of these antibodies, or fragments attached to solid phase substrates. These assays will also allow for the diagnostic determination of the effects of either ligand binding region mutations and modifications, or other mutations and modifications, e.g., which affect signaling or enzymatic function.

This invention also contemplates the use of competitive drug screening assays, e.g., where neutralizing antibodies to the receptor complexes or fragments compete with a test compound for binding to a ligand or other antibody. In this manner, the neutralizing antibodies or fragments can be used to detect the presence of a polypeptide which shares one or more binding sites to a receptor and can also be used to occupy binding sites on a receptor that might otherwise bind a ligand.

V. Nucleic Acids and Protein

The invention provides nucleic acids for therapeutic uses, e.g., nucleic acids encoding IL-17C or DCRS9, or an antigenic fragment thereof, the corresponding anti-sense nucleic acids, and hybridization products thereof. The invention also provides compositions for RNA interference, see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189.

DNA which encodes the protein or fragments thereof can be obtained by chemical synthesis, screening cDNA libraries, or by screening genomic libraries prepared from a wide variety of cell lines or tissue samples. Natural sequences can be isolated using standard methods and the sequences provided herein, e.g., in SEQ ID NOS: 1, 3, 5, 7, 9, 11, 13, 15, and 17. Other species counterparts can be identified by hybridization techniques, or by various PCR techniques, combined with or by searching in sequence databases, e.g., GenBank.

This DNA can be expressed in a wide variety of host cells for the synthesis of a full-length receptor or fragments which can in turn, for example, be used to generate polyclonal or monoclonal antibodies; for binding studies; for construction and expression of modified ligand binding or kinase/phosphatase domains; and for structure/function studies. Variants or fragments can be expressed in host cells that are transformed or transfected with appropriate expression vectors. These molecules can be substantially free of protein or cellular contaminants, other than those derived from the recombinant host, and therefore are particularly useful in pharmaceutical compositions when combined with a pharmaceutically acceptable carrier and/or diluent. The protein, or portions thereof, may be expressed as fusions with other proteins. Combinations of the described proteins, or nucleic acids encoding them, are particularly interesting.

Expression vectors are typically self-replicating DNA or RNA constructs containing the desired receptor gene or its fragments, usually operably linked to suitable genetic control elements that are recognized in a suitable host cell. These control elements are capable of effecting expression within a suitable host. The multiple genes may be coordinately expressed, and may be on a polycistronic message. The specific type of control elements necessary to effect expression will depend upon the eventual host cell used. Generally, the genetic control elements can include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also usually contain an origin of replication that allows the vector to replicate independently of the host cell.

The vectors of this invention include those which contain DNA which encodes a combination of proteins, as described, or a biologically active equivalent polypeptide. The DNA can be under the control of a viral promoter and can encode a selection marker. This invention further contemplates use of such expression vectors which are capable of expressing eukaryotic cDNAs coding for such proteins in a prokaryotic or eukaryotic host, where the vector is compatible with the host and where the eukaryotic cDNAs are inserted into the vector such that growth of the host containing the vector expresses the cDNAs in question. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the protein or its fragments in various hosts using vectors that do not contain a replication origin that is recognized by the host cell. It is also possible to use vectors that cause integration of the protein encoding portions into the host DNA by recombination.

Vectors, as used herein, comprise plasmids, viruses, bacteriophage, integratable DNA fragments, and other vehicles which enable the integration of DNA fragments into the genome of the host. Expression vectors are specialized vectors which contain genetic control elements that effect expression of operably linked genes. Plasmids are the most commonly used form of vector but all other forms of vectors which serve an equivalent function and which are, or become, known in the art are suitable for use herein. See, e.g., Pouwels, et al. (1985 and Supplements) Cloning Vectors: A Laboratory Manual, Elsevier, N.Y., and Rodriguez, et al. (eds.) (1988) Vectors: A Survey of Molecular Cloning Vectors and Their Uses, Buttersworth, Boston.

Transformed cells are cells, preferably mammalian, that have been transformed or transfected with vectors constructed using recombinant DNA techniques. Transformed host cells usually express the desired proteins, but for purposes of cloning, amplifying, and manipulating its DNA, do not need to express the subject proteins. This invention further contemplates culturing transformed cells in a nutrient medium, thus permitting the proteins to accumulate. The proteins can be recovered, either from the culture or, in certain instances, from the culture medium.

For purposes of this invention, nucleic sequences are operably linked when they are functionally related to each other. For example, DNA for a presequence or secretory leader is operably linked to a polypeptide if it is expressed as a preprotein or participates in directing the polypeptide to the cell membrane or in secretion of the polypeptide. A promoter is operably linked to a coding sequence if it controls the transcription of the polypeptide; a ribosome binding site is operably linked to a coding sequence if it is positioned to permit translation. Usually, operably linked means contiguous and in reading frame, however, certain genetic elements such as repressor genes are not contiguously linked but still bind to operator sequences that in turn control expression.

Suitable host cells include prokaryotes, lower eukaryotes, and higher eukaryotes. Prokaryotes include both gram negative and gram positive organisms, e.g., E. coli and B. subtilis. Lower eukaryotes include yeasts, e.g., S. cerevisiae and Pichia, and species of the genus Dictyostelium. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express the receptor or its fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac (pDR540). See Brosius, et al. (1988) “Expression Vectors Employing Lambda-, trp-, lac-, and Ipp-derived Promoters”, in Vectors: A Survey of Molecular Cloning Vectors and Their Uses (eds. Rodriguez and Denhardt), Buttersworth, Boston, Chapter 10, pp. 205-236.

Lower eukaryotes, e.g., yeasts and Dictyostelium, may be transformed with DCRS8 sequence containing vectors. For purposes of this invention, the most common lower eukaryotic host is the baker's yeast, Saccharomyces cerevisiae. It will be used to generically represent lower eukaryotes although a number of other strains and species are also available. Yeast vectors typically consist of a replication origin (unless of the integrating type), a selection gene, a promoter, DNA encoding the receptor or its fragments, and sequences for translation termination, polyadenylation, and transcription termination. Suitable expression vectors for yeast include such constitutive promoters as 3-phosphoglycerate kinase and various other glycolytic enzyme gene promoters or such inducible promoters as the alcohol dehydrogenase 2 promoter or metallothionine promoter. Suitable vectors include derivatives of the following types: self-replicating low copy number (such as the YRp-series), self-replicating high copy number (such as the YEp-series); integrating types (such as the YIp-series), or mini-chromosomes (such as the YCp-series).

Higher eukaryotic tissue culture cells are normally the preferred host cells for expression of the functionally active interleukin or receptor proteins. In principle, many higher eukaryotic tissue culture cell lines are workable, e.g., insect baculovirus expression systems, whether from an invertebrate or vertebrate source. However, mammalian cells are preferred. Transformation or transfection and propagation of such cells has become a routine procedure. Examples of useful cell lines include HeLa cells, Chinese hamster ovary (CHO) cell lines, baby rat kidney (BRK) cell lines, insect cell lines, bird cell lines, and monkey (COS) cell lines. Expression vectors for such cell lines usually include an origin of replication, a promoter, a translation initiation site, RNA splice sites (if genomic DNA is used), a polyadenylation site, and a transcription termination site. These vectors also usually contain a selection gene or amplification gene. Suitable expression vectors may be plasmids, viruses, or retroviruses carrying promoters derived, e.g., from such sources as from adenovirus, SV40, parvoviruses, vaccinia virus, or cytomegalovirus. Representative examples of suitable expression vectors include pcDNA1; pCD, see Okayama, et al. (1985) Mol. Cell Biol. 5:1136-1142; pMC1neo PolyA, see Thomas, et al. (1987) Cell 51:503-512; and a baculovirus vector such as pAC 373 or pAC 610.

For secreted proteins and some membrane proteins, an open reading frame usually encodes a polypeptide that consists of a mature or secreted product covalently linked at its N-terminus to a signal peptide. The signal peptide is cleaved prior to secretion of the mature, or active, polypeptide. The cleavage site can be predicted with a high degree of accuracy from empirical rules, e.g., von-Heijne (1986) Nucleic Acids Research 14:4683-4690; and Nielsen, et al. (1997) Protein Eng. 10: 1-12, and the precise amino acid composition of the signal peptide often does not appear to be critical to its function, e.g., Randall, et al. (1989) Science 243:1156-1159; and Kaiser, et al. (1987) Science 235:312-317. The mature proteins of the invention can be readily determined using standard methods.

It will often be desired to express these polypeptides in a system which provides a specific or defined glycosylation pattern. In this case, the usual pattern will be that provided naturally by the expression system. However, the pattern will be modifiable by exposing the polypeptide, e.g., an unglycosylated form, to appropriate glycosylating proteins introduced into a heterologous expression system. For example, the receptor gene may be co-transformed with one or more genes encoding mammalian or other glycosylating enzymes. Using this approach, certain mammalian glycosylation patterns will be achievable in prokaryote or other cells. Expression in prokaryote cells will typically lead to unglycosylated forms of protein.

The source of DCRS8 can be a eukaryotic or prokaryotic host expressing recombinant DCRS8, such as is described above. The source can also be a cell line, but other mammalian cell lines are also contemplated by this invention, with the preferred cell line being from the human species.

Now that the sequences are known, the primate IL-17C (SEQ ID NOs:23, 24) or DCRS9 (SEQ ID NOs:11, 12), fragments, or derivatives thereof can be prepared by conventional processes for synthesizing peptides. These include processes such as are described in Stewart and Young (1984) Solid Phase Peptide Synthesis, Pierce Chemical Co., Rockford, Ill.; Bodanszky and Bodanszky (1984) The Practice of Peptide Synthesis, Springer-Verlag, New York; and Bodanszky (1984) The Principles of Peptide Synthesis, Springer-Verlag, New York. For example, an azide process, an acid chloride process, an acid anhydride process, a mixed anhydride process, an active ester process (for example, p-nitrophenyl ester, N-hydroxysuccinimide ester, or cyanomethyl ester), a carbodiimidazole process, an oxidative-reductive process, or a dicyclohexylcarbodiimide (DCCD)/additive process can be used. Solid phase and solution phase syntheses are both applicable to the foregoing processes. Similar techniques can be used with partial IL-17C or DCRS9 sequences.

The IL-17C and DCRS9 proteins, fragments, or derivatives are suitably prepared in accordance with the above processes as typically employed in peptide synthesis, generally either by a so-called stepwise process which comprises condensing an amino acid to the terminal amino acid, one by one in sequence, or by coupling peptide fragments to the terminal amino acid. Amino groups that are not being used in the coupling reaction typically must be protected to prevent coupling at an incorrect location.

If a solid phase synthesis is adopted, the C-terminal amino acid is bound to an insoluble carrier or support through its carboxyl group. The insoluble carrier is not particularly limited as long as it has a binding capability to a reactive carboxyl group. Examples of such insoluble carriers include halomethyl resins, such as chloromethyl resin or bromomethyl resin, hydroxymethyl resins, phenol resins, tert-alkyloxycarbonylhydrazidated resins, and the like.

An amino group-protected amino acid is bound in sequence through condensation of its activated carboxyl group and the reactive amino group of the previously formed peptide or chain, to synthesize the peptide step by step. After synthesizing the complete sequence, the peptide is split off from the insoluble carrier to produce the peptide. This solid-phase approach is generally described by Merrifield, et al. (1963) J. Am. Chem. Soc. 85:2149-2156.

The prepared protein and fragments thereof can be isolated and purified from the reaction mixture by means of peptide separation, e.g., by extraction, precipitation, electrophoresis, various forms of chromatography, and the like. The receptors of this invention can be obtained in varying degrees of purity depending upon desired uses. Purification can be accomplished by use of the protein purification techniques disclosed herein, see below, or by the use of the antibodies herein described in methods of immunoabsorbant affinity chromatography. This immunoabsorbant affinity chromatography is carried out by first linking the antibodies to a solid support and then contacting the linked antibodies with solubilized lysates of appropriate cells, lysates of other cells expressing the receptor, or lysates or supernatants of cells producing the protein as a result of DNA techniques, see below.

Generally, the purified protein will be at least about 40% pure, ordinarily at least about 50% pure, usually at least about 60% pure, typically at least about 70% pure, more typically at least about 80% pure, preferable at least about 90% pure and more preferably at least about 95% pure, and in particular embodiments, 97%-99% or more. Purity will usually be on a weight basis, but can also be on a molar basis. Different assays will be applied as appropriate. Individual proteins may be purified and thereafter combined.

VI. Antibodies

Antibodies, and fragments and derivatives thereof, that specifically bind to IL-17C or DCRS9 can be prepared by immunization with antigenic fragments. Regions of IL-17C (SEQ ID NO:23) having increased immunogenicity or antigenicity include, e.g., amino acids 26-107 of SEQ ID NO:23 (segment beginning, GHPHSH . . . ); amino acids 133-144 (GCIDAR . . . ); amino acids 20-41 (HDPSLR . . . ); amino acids 68-89 (SSLEAA . . . ); amino acids 98-105 (EADTHQ . . . ); amino acid 111-123 (YRVDTD . . . ); amino acids 136-144 (DARTG . . . ); and amino acids 159-169 (RRRPCS . . . ) of SEQ ID NO:23.

Regions of DCRS9 (SEQ ID NO:12) having increased immunogenicity include, e.g., amino acids 263-324 of SEQ ID NO:12 (segment beginning HFTDY . . . ); amino acids 397-494 (VSQVW . . . ); amino acids 518-610 (EQGTVL . . . ); amino acids 65-92 (GASSTS . . . ); amino acids 142-155 (THKGME . . . ); and amino acids 197-219 (ECEELS . . . ); of SEQ ID NO:12. Antigenic regions were determined by according to Parker, et al. (1986) Biochemistry 25:5425-5432 and Welling, et al. (1985) FEBS Lett. 188:215-218, optionally with use of software from Vector NTI® Suite (Informax, Inc., Bethesda, Md.).

Purification of antigen is not necessary for the generation of antibodies. Immunization can be performed by DNA vector immunization, see, e.g., Wang, et al. (1997) Virology 228: 278-284. Alternatively, animals can be immunized with cells or cell membranes bearing the antigen of interest followed by hybridoma production (see, e.g., Meyaard, et al. (1997) Immunity 7:283-290; Wright, et al. (2000) Immunity 13:233-242; Preston, et al. (1997) Eur. J Immunol. 27:1911-1918; Kaithamana, et al. (1999) New Engl. J Med. 163:5157-5164).

Humanized antibodies, chimeric antibodies, single chain antibodies, single domain antibodies, bispecific antibodies, and peptide mimetics of antibodies are described (see, e.g., Maynard and Georgiou (2000) Annu. Rev. Biomed. Eng. 2:339-376; Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) New Engl. J. Med. 148:1547-1553; Casset, et al. (2002) Biochem. Biophys. Res. Commun. 307:198-205; U.S. Pat. Nos. 5,932, 448; 5,532,210; 6,129,914; 6,133,426; 4,946,778).

Antibodies can be raised to the various mammalian, e.g., primate IL-17C or DCRS9 proteins and fragments thereof, both in naturally occurring native forms and in their recombinant forms, the difference being that antibodies to the active receptor are more likely to recognize epitopes which are only present in the native conformations. Denatured antigen detection can also be useful in, e.g., Western analysis. Anti-idiotypic antibodies are also contemplated, which would be useful as agonists or antagonists of a natural receptor or an antibody.

Antibodies to IL-17C (SEQ ID NOs:23, 24) or to DCRS9 (SEQ ID NOs:11, 12) will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰ M, and most preferably at least 10⁻¹¹ M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

The antibodies, including antigen binding fragments, of this invention can have significant diagnostic or therapeutic value. They can be potent antagonists that bind to the receptor and inhibit binding to ligand or inhibit the ability of the receptor to elicit a biological response, e.g., act on its substrate. They also can be useful as non-neutralizing antibodies and can be coupled to toxins or radionuclides to bind producing cells, or cells localized to the source of the interleukin. Further, these antibodies can be conjugated to drugs or other therapeutic agents, either directly or indirectly by means of a linker.

The antibodies of this invention can also be useful in diagnostic applications. As capture or non-neutralizing antibodies, they might bind to the receptor without inhibiting ligand or substrate binding. As neutralizing antibodies, they can be useful in competitive binding assays. They will also be useful in detecting or quantifying ligand. They may be used as reagents for Western blot analysis, or for immunoprecipitation or immunopurification of the respective protein. Likewise, nucleic acids and proteins may be immobilized to solid substrates for affinity purification or detection methods. The substrates may be, e.g., solid resin beads or sheets of plastic.

Protein fragments may be joined to other materials, particularly polypeptides, as fused or covalently joined polypeptides to be used as immunogens. Mammalian cytokine receptors and fragments may be fused or covalently linked to a variety of immunogens, such as keyhole limpet hemocyanin, bovine serum albumin, tetanus toxoid, etc. See (1969) Microbiology, Hoeber Medical Division, Harper and Row; Landsteiner (1962) Specificity of Serological Reactions, Dover Publications, New York; and Williams, et al. (1967) Methods in Immunology and Immunochemistry, Vol. 1, Academic Press, New York, for descriptions of methods of preparing polyclonal antisera. A typical method involves hyperimmunization of an animal with an antigen. The blood of the animal is then collected shortly after the repeated immunizations and the gamma globulin is isolated.

In some instances, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2nd ed.) Academic Press, New York; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve in vitro exposure of lymphocytes to the antigenic polypeptides or alternatively to selection of libraries of antibodies in phage or similar vectors. See, Huse, et al. (1989) Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents, teaching the use of such labels include U.S. Pat. Nos. 3,817,837; 3,850,752; 3,939,350; 3,996,345; 4,277,437; 4,275,149; and 4,366,241. Also, recombinant or chimeric immunoglobulins may be produced, see Cabilly, U.S. Pat. No. 4,816,567; or made in transgenic mice, see Mendez, et al. (1997) Nature Genetics 15:146-156; Abgenix; and Medarex.

The antibodies of this invention can also be used for affinity chromatography in isolating the L-17C or DCRS9 proteins or peptides. Columns can be prepared where the antibodies are linked to a solid support, e.g., particles, such as agarose, Sephadex®, or the like, where a cell lysate may be passed through Alternatively, the protein may be used to purify antibody. Appropriate cross absorptions or depletions may be applied to the column, the column washed, followed by increasing concentrations of a mild denaturant, whereby the purified protein will be released.

The antibodies may also be used to screen expression libraries for particular expression products. Usually the antibodies used in such a procedure will be labeled with a moiety allowing easy detection of presence of antigen by antibody binding.

Antibodies raised against a cytokine receptor will also be used to raise anti-idiotypic antibodies. These will be useful in detecting or diagnosing various immunological conditions related to expression of the protein or cells which express the protein. They also will be useful as agonists or antagonists of the ligand, which may be competitive inhibitors or substitutes for naturally occurring ligands.

A cytokine receptor protein that specifically binds to or that is specifically immunoreactive with an antibody generated against a defined immunogen, such as an immunogen consisting of the amino acid sequence of SEQ ID NO: 10, is typically determined in an immunoassay. The immunoassay typically uses a polyclonal antiserum which was raised, e.g., to a protein of SEQ ID NO: 10. This antiserum is selected to have low crossreactivity against other cytokine receptor family members, preferably from the same species, and any such crossreactivity is removed by immunoabsorption prior to use in the immunoassay.

In order to produce antisera for use in an immunoassay, the protein, e.g., of IL-17C or DCRS9, is isolated as described herein. For example, recombinant protein may be produced in a mammalian cell line. An appropriate host, e.g., an inbred strain of mice such as Balb/c, is immunized with the selected protein, typically using a standard adjuvant, such as Freund's adjuvant, and a standard mouse immunization protocol (see Harlow and Lane, supra). Alternatively, a synthetic peptide derived from the sequences disclosed herein and conjugated to a carrier protein can be used an immunogen. Polyclonal sera are collected and titered against the immunogen protein in an immunoassay, e.g., a solid phase immunoassay with the immunogen immobilized on a solid support. Polyclonal antisera with a titer of 10⁴ or greater are selected and tested for their cross reactivity against other cytokine receptor family members using a competitive binding immunoassay such as the one described in Harlow and Lane, supra, at pages 570-573. Preferably at least two cytokine receptor family members are used in this determination. These cytokine receptor family members can be produced as recombinant proteins and isolated using standard molecular biology and protein chemistry techniques as described herein.

Immunoassays in the competitive binding format can be used for the crossreactivity determinations. For example, the protein of IL-17C or DCRS9 can be immobilized to a solid support. Proteins added to the assay compete with the binding of the antisera to the immobilized antigen. The ability of the above proteins to compete with the binding of the antisera to the immobilized protein is compared to the other proteins. The percent crossreactivity for the above proteins is calculated, using standard calculations. Those antisera with less than 10% crossreactivity with each of the proteins listed above are selected and pooled. The cross-reacting antibodies are then removed from the pooled antisera by immunoabsorption with the above-listed proteins.

The immunoabsorbed and pooled antisera are then used in a competitive binding immunoassay as described above to compare a second protein to the immunogen protein, e.g. of IL-17C or DCRS9. In order to make this comparison, the two proteins are each assayed at a wide range of concentrations and the amount of each protein required to inhibit 50% of the binding of the antisera to the immobilized protein is determined. If the amount of the second protein required is less than twice the amount of the protein of the selected protein or proteins that is required, then the second protein is said to specifically bind to an antibody generated to the immunogen.

It is understood that these cytokine receptor proteins are members of a family of homologous proteins that comprise at least 9 so far identified members, 6 mammalian and 3 worm embodiments. For a particular gene product, such as L-17C or DCRS9, the term refers not only to the amino acid sequences disclosed herein, but also to other proteins that are allelic, non-allelic, or species variants. It is also understood that the terms include nonnatural mutations introduced by deliberate mutation using conventional recombinant technology such as single site mutation, or by excising short sections of DNA encoding the respective proteins, or by substituting new amino acids, or adding new amino acids. Such minor alterations typically will substantially maintain the immunoidentity of the original molecule and/or its biological activity. Thus, these alterations include proteins that are specifically immunoreactive with a designated naturally occurring IL-17C or DCRS9 protein. The biological properties of the altered proteins can be determined by expressing the protein in an appropriate cell line and measuring the appropriate effect, e.g., upon transfected lymphocytes. Particular protein modifications considered minor would include conservative substitution of amino acids with similar chemical properties, as described above for the cytokine receptor family as a whole. By aligning a protein optimally with the protein of the cytokine receptors and by using the conventional immunoassays described herein to determine immunoidentity, one can determine the protein compositions of the invention.

The invention encompasses methods for using a soluble receptor derived from DCRS9 for use, e.g., as an IL-17C agonist or antagonist or as a DCRS9 agonist or antagonist. The soluble receptor can be used for modulating an activity of a cell, for treating a disorder or pathological condition, or for diagnostic purposes. The soluble receptor can comprise amino acids-23-424 of SEQ ID NO:12, amino acids 1-424 of SEQ ID NO:12, or antigenic fragment thereof. Soluble receptor versions with one or more conservatively modified amino acids, with a truncation from the N-terminal end, with a truncation from the C-terminal end, and/or with one or more internal deletions, are contemplated. Preferably, the soluble receptor will not comprise the transmembrane region (LLILALLALLTLLGVVL; amino acids 424-440 of SEQ ID NO:12).

VII. Kits and Quantitation

Both naturally occurring and recombinant forms of the cytokine receptor like molecules of this invention are particularly useful in kits and assay methods. For example, these methods would also be applied to screening for binding activity, e.g., ligands for these proteins. Several methods of automating assays have been developed in recent years so as to permit screening of tens of thousands of compounds per year. See, e.g., a BIOMEK automated workstation, Beckman Instruments, Palo Alto, Calif., and Fodor, et al. (1991) Science 251:767-773. The latter describes means for testing binding by a plurality of defined polymers synthesized on a solid substrate. The development of suitable assays to screen for a ligand or agonist/antagonist homologous proteins can be greatly facilitated by the availability of large amounts of purified, soluble cytokine receptors in an active state such as is provided by this invention.

Purified protein can be coated directly onto plates for use in the aforementioned ligand screening techniques. However, non-neutralizing antibodies to these proteins can be used as capture antibodies to immobilize the respective receptor on the solid phase, useful, e.g., in diagnostic uses.

This invention also contemplates use of receptor subunit, fragments thereof, peptides, and their fusion products in a variety of diagnostic kits and methods for detecting the presence of the protein or its ligand. Alternatively, or additionally, antibodies against the molecules may be incorporated into the kits and methods. Typically the kit will have a compartment containing, e.g., a IL-17C or DCRS9 peptide or gene segment or a reagent which recognizes one or the other. Typically, recognition reagents, in the case of peptide, would be a receptor or antibody, or in the case of a gene segment, would usually be a hybridization probe.

A preferred kit for determining the concentration of IL-17C or DCRS9 in a sample would typically comprise a labeled compound, e.g., ligand or antibody, having known binding affinity for IL-17C or DCRS9, a source of IL-17C or DCRS9 (naturally occurring or recombinant) as a positive control, and a means for separating the bound from free labeled compound, e.g., a solid phase for immobilizing the IL-17C or DCRS9 in the test sample. Compartments containing reagents, and instructions, will normally be provided. Appropriate nucleic acid or protein containing kits are also provided.

Antibodies, including antigen binding fragments, specific for mammalian IL-17C (SEQ ID NO:24) or DCRS9 (SEQ ID NO:12) or a peptide fragment, or receptor fragments are useful in diagnostic applications to detect the presence of elevated levels of ligand and/or its fragments. Diagnostic assays may be homogeneous (without a separation step between free reagent and antibody-antigen complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA) and the like. For example, unlabeled antibodies can be employed by using a second antibody which is labeled and which recognizes the antibody to a cytokine receptor or to a particular fragment thereof. These assays have also been extensively discussed in the literature. See, e.g., Harlow and Lane, supra, and Coligan (ed.) (1991 and periodic supplements) Current Protocols In Immunology Greene/Wiley, New York.

Anti-idiotypic antibodies may have similar use to serve as agonists or antagonists of cytokine receptors. These should be useful as therapeutic reagents under appropriate circumstances.

Frequently, the reagents for diagnostic assays are supplied in kits, so as to optimize the sensitivity of the assay. For the subject invention, depending upon the nature of the assay, the protocol, and the label, either labeled or unlabeled antibody, or labeled ligand is provided. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent, and will contain instructions for proper use and disposal of reagents. Desirably, the reagents are provided as a dry lyophilized powder, where the reagents may be reconstituted in an aqueous medium having appropriate concentrations for performing the assay.

The aforementioned constituents of the diagnostic assays may be used without modification or may be modified in a variety of ways. For example, labeling may be achieved by covalently or non-covalently joining a moiety which directly or indirectly provides a detectable signal. In many of these assays, a test compound, cytokine receptor, or antibodies thereto can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups.

There are also numerous methods of separating the bound from the free ligand, or alternatively the bound from the free test compound. The cytokine receptor can be immobilized on various matrixes followed by washing. Suitable matrices include plastic such as an ELISA plate, filters, and beads. Methods of immobilizing the receptor to a matrix include, without limitation, direct adhesion to plastic, use of a capture antibody, chemical coupling, and biotin-avidin. The last step in this approach involves the precipitation of antibody/antigen complex by any of several methods including those utilizing, e.g., an organic solvent such as polyethylene glycol or a salt such as ammonium sulfate. Other suitable separation techniques include, without limitation, the fluorescein antibody magnetizable particle method described in Rattle, et al. (1984) Clin. Chem. 30(9):1457-1461, and the double antibody magnetic particle separation as described in U.S. Pat. No. 4,659,678.

The methods for linking protein or fragments to various labels have been extensively reported in the literature and do not require detailed discussion here. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Another diagnostic aspect of this invention involves use of oligonucleotide or polynucleotide sequences taken from the sequence of a cytokine receptor. These sequences can be used as probes for detecting levels of the respective cytokine receptor in patients suspected of having an immunological disorder. The preparation of both RNA and DNA nucleotide sequences, the labeling of the sequences, and the preferred size of the sequences has received ample description and discussion in the literature. Normally an oligonucleotide probe should have at least about 14 nucleotides, usually at least about 18 nucleotides, and the polynucleotide probes may be up to several kilobases. Various labels may be employed, most commonly radionuclides, particularly ³²P. However, other techniques may also be employed, such as using biotin modified nucleotides for introduction into a polynucleotide. The biotin then serves as the site for binding to avidin or antibodies, which may be labeled with a wide variety of labels, such as radionuclides, fluorescers, enzymes, or the like. Alternatively, antibodies may be employed which can recognize specific duplexes, including DNA duplexes, RNA duplexes, DNA-RNA hybrid duplexes, or DNA-protein duplexes. The antibodies in turn may be labeled and the assay carried out where the duplex is bound to a surface, so that upon the formation of duplex on the surface, the presence of antibody bound to the duplex can be detected. The use of probes to the novel RNA may be carried out in conventional techniques such as nucleic acid hybridization, plus and minus screening, recombinational probing, hybrid released translation (HRT), and hybrid arrested translation (HART). Antisense nucleic acids, which may be used to block protein expression, are also provided. See, e.g., Isis Pharmaceuticals, Sequitur, Inc., or Hybridon. This also includes amplification techniques such as polymerase chain reaction (PCR).

Diagnostic kits which also test for the qualitative or quantitative presence of other markers are also contemplated. Diagnosis or prognosis may depend on the combination of multiple indications used as markers. Thus, kits may test for combinations of markers. See, e.g., Viallet, et al. (1989) Progress in Growth Factor Res. 1:89-97.

VIII. Therapeutic Utility

The invention provides agonists and antagonists of IL-17C, e.g., a binding composition specific for a polypeptide or nucleic acid of IL-17C, for the treatment or diagnosis of inflammation, autoimmune disorders, psoriasis, atopic dermatitis, inflammatory bowel disorders (IBD), e.g., Crohn's disease or ulcerative colitis, asthma or allergy, interstitial lung disorder, chronic pulmonary obstructive disorder (COPD), multiple sclerosis, systemic lupus erythematosus(SLE), rheumatoic arthritis, diabetes mellitus, transplant rejection, graft versus host disorder (GVHD), sepsis, atherosclerosis, and proliferative conditions, e.g., cancer, tumors, dysplasia, metastasis, and angiogenesis.

The invention provides agonists and antagonists of IL-17C or DCRS9, e.g., a binding composition specific for a polypeptide or nucleic acid of IL-17C, for the treatment or diagnosis of inflammation, autoimmune disorders, psoriasis, atopic dermatitis, inflammatory bowel disorders (IBD), e.g., Crohn's disease or ulcerative colitis, asthma or allergy, interstitial lung disorder, chronic pulmonary obstructive disorder (COPD), multiple sclerosis, systemic lupus erythematosus(SLE), rheumatoic arthritis, diabetes mellitus, atherosclerosis, transplant rejection, graft versus host disorder (GVHD), sepsis, and proliferative conditions, e.g., cancer, tumors, dysplasia, metastasis, and angiogenesis.

IL-17C (SEQ ID NOs:23 or 24) and DCRS9 (SEQ ID NOs:11 or 12) expression both increase in psoriasis. Psoriasis, a common disorder affecting about 2% of the world's population, involves scaling of the skin and pustular lesions. Of the psoriasis patients in the United States, about one million require ultraviolet or immunosuppressive therapy. About 10% of patients with psoriasis also develop psoriatic arthritis, a debilitating condition. Psoriasis involves hyperproliferation of keratinocytes and infiltration of white blood cells in the skin. The inflammation of psoriasis is mediated by, e.g., T cells, monocytes and macrophages, neutrophils, mast cells, and antigen presenting cells (APCs) such as dendritic cells and Langerhans cells.

IL-2, IFNgamma, TNFbeta, IL-5, and other cytokines, contribute to the keratinocyte hyperproliferation. Innate response, e.g., involving bacterial lipopolysaccharide (LPS; glycolipid), has been implicated as part of the etiology of psoriasis (see, e.g., Bos and De Rie (1999) Immunology Today 20:40-46; Ellis, et al. (2001) New Engl. J. Med. 345:248-255; Bhalerao and Bowcock (1998) Human Mol. Genetics 7:1537-1545; van de Kerkhof (2000) Clin. Exp. Dermatol. 25:165; Tanaka, et al. (2000) Brit. J Dermatol. 143:728-732; Nickoloff (1999) J. Clin. Invest. 104:1161-1164; Curry, et al. (2003) Arch. Pathol. Lab. Med. 127:178-186; Travers, et al. (1999) J. Clin. Invest. 104:1181-1189; Greaves and Weinstein (1995) New Engl. J. Med. 332:581-588; Robert and Kupper (1999) New Engl. J. Med. 341:1817-1828; Bos and De Rie (1999) Immunol. Today 20:40-46), Shimizu, et al. (2002) Histochem. Cell Biol. 118:251-257, Gottleib, et al. (1995) Nature Med. 1:442-447, Abrams, et al. (2000) J. Exp. Med. 192:681-693; Yu, et al. (2002) Dermatology 204:94-99). Psoriatic arthritis, atopic dermatitis, and asthma are associated with psoriasis (McInnes, et al. (2001) J. Immunol. 176:4075-4082; Welp, et al. (1989) Hautarzt 40:496-500).

DCRS9 expression increases in IBD, e.g., Crohn's disease, as well as in the colon of the mouse IL-10 knockout model for IBD (Madsen (2001) Clin. Invest. Med. 24:250-257). IBD, which encompasses the chronic, disabling conditions that include Crohn's disease (CD) and ulcerative colitis (UC), affecting about one million people in the United States, involves infiltration by lymphocytes and neutrophils. The pathology of this disorder involves increased inflammatory response to the gut microflora, i.e., an abnormal T cell immune response. Cytokines mediate IBD, as model studies with animals have shown that treatment with IL-10, anti-IL-12 antibodies, anti-interferon-gamma, or soluble IL-6 receptor, result in relief from the disorder. Anti-tumor necrosis factor (TNF) antibodies are effective in treating human IBD, resulting in healing of mucosal lesions and reduction of infiltrates of lymphocytes and neutrophils. The transcription factor NFκB plays a central role in the inflammation of Crohn's disease and ulcerative colitis (see, e.g., Ardizzone and Porro (2002) J. Int. Medicine 252:475-496; Groux and Powrie (1999) Immunol. Today 20:442-445; Pizarro, et al. (2003) Trends Mol. Medicine 9:218-222; Elson (2002) New Engl. J. Med. 346:614-616; Podolsky (2002) New Engl. J. Med. 347:417-429; Madsen (2002) Gastroenterol. 123:2140-2144; Segain, et al. (2000) Gut 47:397-403; Loncar, et al. (2003) Gut 52:1297-1303).

DCRS9 expression increases in response to infection with the helminth, Nippostrongylus. Exposure to Nippostrongylus, or other helminths, has been found to contribute to or be associated with gut inflammation, asthma, allergies, pulmonary-inflammation, and increased airway hyperresponsiveness (see, e.g., Takeyama, et al. (1997) J. Gastroenterol. Hepatol. 12:204-206; Bundy (1986) Trans. R. Soc. Trop. Med. Hyg. 80:706-718; Tanaka, et al. (1983) Parasitology 86(Pt.2):291-300; Uston, et al. (2003) World J. Gastroenterol. 9:1834-1835; Waters, et al. (1999) J. Parasitol. 85:1100-1105; Gay, et al. (2000) Neuroimmunomodulation 8:171-178; Gay, et al. (2001) Neurogastroenterol. Motil. 13:155-162; Faussone-Pellegrini, et al. (2002) Neurogastroenterol. Motil. 14:83-95; Coyle, et al. (1998) Eur. J. Immunol. 28:2640-2647; Moqbel, et al. (1986) J. Immunol. 137:296-301).

DCRS9 expression also was found to increase in a mouse model for atherosclerosis. Atherosclerosis is an inflammatory disorder, mediated by cytokings and immune cells such as macrophages, and treatable with cytokine agonists or antagonists (see, e.g., IL-10 (Ross (2003) New Engl. J. Med. 340:115-126; Ito and Ikeda (2003) Curr. Drugs Targets Inflamm. Allergy 2:257-265; von der Thusen, et al. (2003) Pharmacol. Rev. 55:133-166).

IL-17C expression increases in airway epithelial cells treated with IL-4, IL-13, and TNFalpha. Epithelial cells, e.g., when exposed to IL-4, IL-13, and/or TNFalpha, contributed to the pathology lung disorders, such as allergy, asthma, lung inflammation, and airway hyperresponsiveness (see, e.g., Laoukili, et al. (2001) J. Clin. Invest. 108:1817-1824; Lilly, et al. (1997) J. Clin. Invest. 99:1767-1773; Kumar, et al. (2002) Clin. Exp. Allergy 32:1104-1111; Venkayya, et al. (2002) Am. J. Respir. Cell Mol. Biol. 26:202-208; Izuhara (2003) Clin. Chem. Lab Med. 41:860-864; Richter, et al. (2001) Am. J. Respir. Cell Mol. Biol. 25:385-391; Relova, et al. (2001) Cell Biol. International 25:563-566).

The present invention provides methods to treat interstitial lung disorders, e.g., idiopathic pulmonary fibrosis, eosinophilic granuloma, or hypersensitivity pneumonitis. Expression of both IL-17C and DCRS9 increase in interstitial lung disorder, e.g., lung hypersensitivity pneumonitis. Idiopathic pulmonary fibrosis, which has a grim prognosis, involves activated alveolar epithelial cells, fibroblastic foci, and deposit of extracellular matrix. Inflammation occurs, but the major feature is fibroblastic foci (see, e.g., Kamp (2003) Chest 124:1187-1189; White, et al. (2003) J. Pathol. 201:343-354). Pulmonary eosinophilic granuloma is a localized nonmalignant histiocytosis. It can resolve, or progress to a fibrotic stage. The disorder is associated with smoking (see, e.g., Levine and Nickelleit (1994) New Engl. J Med. 330:347-353; Rajagopol and Mark (2002) New Engl. J. Med. 347:1262-1268; Miadonna, et al. (2000) Monaldi Arch Chest Dis. 55:3-5). Hypersensitivity pneumonitis (a.k.a. extrinsic allergic alveolitis), caused by inhaled allergens, involves inflammation in peripheral airways and surrounding interstitial tissues. Monocytes accumulate and mature into foamy macrophages that develop into granulomas. The disorder also involves bronchiolitis, interstitial lymphocyte infiltration, and may include a “honeycombed lung” fibrosis (see, e.g., Patel, et al. (2001) J. Allergy Clin. Immunol. 108:661-670; Yi (2002) Crit. Rev. Clin. Lab. Sci. 39:581-629).

This invention provides reagents with significant therapeutic value. See, e.g., Levitzki (1996) Curr. Opin. Cell Biol. 8:239-244. The cytokine receptors (naturally occurring or recombinant), fragments thereof, mutein receptors, and antibodies, along with compounds identified as having binding affinity to the receptors or antibodies, should be useful in the treatment of conditions exhibiting abnormal expression of the receptors of their ligands. Such abnormality will typically be manifested by immunological disorders, e.g., innate immunity, or developmentally. Additionally, this invention should provide therapeutic value in various diseases or disorders associated with abnormal expression or abnormal triggering of response to the ligand. For example, the IL-1 ligands have been suggested to be involved in morphologic development, e.g., dorso-ventral polarity determination, and immune responses, particularly the primitive innate responses. See, e.g., Sun, et al. (1991) Eur. J. Biochem. 196:247-254; and Hultmark (1994) Nature 367:116-117.

Recombinant cytokine receptors, muteins, agonist or antagonist antibodies thereto, or antibodies can be purified and then administered to a patient. These reagents can be combined for therapeutic use with additional active ingredients, e.g., in conventional pharmaceutically acceptable carriers or diluents, along with physiologically innocuous stabilizers and excipients. These combinations can be sterile, e.g., filtered, and placed into dosage forms as by lyophilization in dosage vials or storage in stabilized aqueous preparations. This invention also contemplates use of antibodies or binding fragments thereof which are not complement binding.

Ligand screening using cytokine receptor or fragments thereof can be performed to identify molecules having binding affinity to the receptors. Subsequent biological assays can then be utilized to determine if a putative ligand can provide competitive binding, which can block intrinsic stimulating activity. Receptor fragments can be used as a blocker or antagonist in that it blocks the activity of ligand. Likewise, a compound having intrinsic stimulating activity can activate the receptor and is thus an agonist in that it simulates the activity of ligand, e.g., inducing signaling. This invention further contemplates the therapeutic use of antibodies to cytokine receptors as antagonists.

The quantities of reagents necessary for effective therapy will depend upon many different factors, including means of administration, target site, reagent physiological life, pharmacological life, physiological state of the patient, and other medicants administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Penn. Methods for administration are discussed therein and below, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Because of the likely high affinity binding, or turnover numbers, between a putative ligand and its receptors, low dosages of these reagents would be initially expected to be effective. And the signaling pathway suggests extremely low amounts of ligand may have effect. Thus, dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or slow release apparatus will often be utilized for continuous administration.

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg, see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

Cytokine receptors, fragments thereof, and antibodies or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in many conventional dosage formulations. While it is possible for the active ingredient to be administered alone, it is preferable to present it as a pharmaceutical formulation. Formulations comprise at least one active ingredient, as defined above, together with one or more acceptable carriers thereof. Each carrier must be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by methods well known in the art of pharmacy. See, e.g., Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Penn.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, MarcelDekker, NY; and Lieberman, et al. (eds.) (1990) Pharmaceutical Dosage Forms: Disperse Systems, Marcel Dekker, NY. The therapy of this invention may be combined with or used in association with other therapeutic agents, particularly agonists or antagonists of other cytokine receptor family members.

IX. Screening

Drug screening using IL-17C (SEQ ID NOs:23, 24) or DCRS9 (SEQ ID NOs:11, 12) or fragments thereof can be performed to identify compounds having binding affinity to the receptor subunit, including isolation of associated components. Subsequent biological assays can then be utilized to determine if the compound has intrinsic stimulating activity and is therefore a blocker or antagonist in that it blocks the activity of the ligand. Likewise, a compound having intrinsic stimulating activity can activate the receptor and is thus an agonist in that it simulates the activity of a cytokine ligand. This invention further contemplates the therapeutic use of antibodies to the receptor as cytokine agonists or antagonists.

Similarly, complexes comprising multiple proteins may be used to screen for ligands or reagents capable of recognizing the complex. Most cytokine receptors comprise at least two subunits, which may be the same, or distinct. Alternatively, the transmembrane receptor may bind to a complex comprising a cytokine-like ligand associated with another soluble protein serving, e.g., as a second receptor subunit.

One method of drug screening utilizes eukaryotic or prokaryotic host cells which are stably transformed with recombinant DNA molecules expressing the IL-17C or DCRS9 in combination with another cytokine receptor subunit. Cells may be isolated which express a receptor in isolation from other functional receptors. Such cells, either in viable or fixed form, can be used for standard antibody/antigen or ligand/receptor binding assays. See also, Parce, et al. (1989) Science 246:243-247; and Owicki, et al. (1990) Proc. Natl. Acad. Sci. USA 87:4007-4011, which describe sensitive methods to detect cellular responses. Competitive assays are particularly useful, where the cells (source of putative ligand) are contacted and incubated with a labeled receptor or antibody having known binding affinity to the ligand, such as ¹²⁵I -antibody, and a test sample whose binding affinity to the binding composition is being measured. The bound and free labeled binding compositions are then separated to assess the degree of ligand binding. The amount of test compound bound is inversely proportional to the amount of labeled receptor binding to the known source. Many techniques can be used to separate bound from free ligand to assess the degree of ligand binding. This separation step could typically involve a procedure such as adhesion to filters followed by washing, adhesion to plastic followed by washing, or centrifugation of the cell membranes. Viable cells could also be used to screen for the effects of drugs on cytokine mediated functions, e.g., second messenger levels, e.g., Ca⁺⁺; cell proliferation; inositol phosphate pool changes; and others. Some detection methods allow for elimination of a separation step, e.g., a proximity sensitive detection system. Calcium sensitive dyes will be useful for detecting Ca⁺⁺ levels, with a fluorimeter or a fluorescence cell sorting apparatus.

X. Ligands

The descriptions of DCRS9 herein provides means to identify ligands, as described above. Such ligand should bind specifically to the respective receptor with reasonably high affinity. Various constructs are made available which allow either labeling of the receptor to detect its ligand. For example, directly labeling cytokine receptor, fusing onto it markers for secondary labeling, e.g., FLAG or other epitope tags, etc., will allow detection of receptor. This can be histological, as an affinity method for biochemical purification, or labeling or selection in an expression cloning approach. A two-hybrid selection system may also be applied making appropriate constructs with the available cytokine receptor sequences. See, e.g., Fields and Song (1989) Nature 340:245-246. Most likely candidates will be structually related to members of the IL-17 family. See, e.g., U.S. Ser. No. 09/480,287.

The broad scope of this invention is best understood with reference to the following examples, which are not intended to limit the inventions to the specific embodiments.

### EXAMPLES

I. General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2nd ed.), vols. 1-3, CSH Press, NY; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Coligan, et al. (ed. 1996) and periodic supplements, Current Protocols In Protein Science, Greene/Wiley, New York; Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Hercules, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System, QIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank and others.

Many techniques applicable to IL-1O receptors may be applied to the DCRSs, as described, e.g., in U.S. Ser. No. 08/110,683 (IL-10 receptor).

II. Computational Analysis

Human sequences related to cytokine receptors were identified from genomic sequence database using, e.g., the BLAST server (Altschul, et al. (1994) Nature Genet. 6:119-129). Standard analysis programs may be used to evaluate structure, e.g., PHD (Rost and Sander (1994) Proteins 19:55-72) and DSC (King and Sternberg (1996) Protein Sci. 5:2298-2310). Standard comparison software includes, e.g., Altschul, et al. (1990) J. Mol. Biol. 215:403-10; Waterman (1995) Introduction to Computational Biology: Maps, Sequences, and Genomes, Chapman & Hall; Lander and Waterman (eds.) (1995) Calculating the Secrets of Life: Applications of the Mathematical Sciences in Molecular Biology, National Academy Press; and Speed and Waterman (eds.) (1996) Genetic Mapping and DNA Sequencing (IMA Volumes in Mathematics and Its Applications, Vol 81) Springer Verlag.

III. Identification of IL-17C as the Ligand of DCRS9.

IL-17C (a.k.a. IL-71; IL-171) was identified as the ligand of DCRS9 (a.k.a. IL-17RE) by procedures detailed below. The mature coding sequences of IL-17 (a.k.a. CTLA-8), IL-17C, IL-17D (a.k.a. IL-73), IL-17E (a.k.a. IL-25; IL-74), and IL-17F were subcloned into the pCMV-1 vector. This vector contains the pre-protrypsin leader followed by the FLAG peptide tag followed by the mature IL-17 ligand sequence. Ten 150 mM dishes of 293T cells in 30 ml DMEM 10% FCS were transfected with 28 ug per plate of pCMV1FlagIL-17, pCMV1FlagIL-17C, pCMV1FlagIL-17D, pCMV1FlagIL-17E, or pCMV1FlagIL-17F, and 56 μl Fugene6 (Roche) and incubated for 48-72 hours and supernatant was collected. Expression of FLAG-tagged ligands was verified by western blotting with ANTI-FLAG® M2 antibody (Sigma, St. Louis, Mo.).

Chimeric receptors were prepared and expressed in cells. The extracellular domains of the IL-17 receptors A-E (IL-17RA; IL-17RB; IL-17RC; IL-17RD; IL-17RE) were subcloned to the transmembrane and intracellular domains of murine GCSFR in the vector pMX-puromycin. Stable cell lines expressing the chimeric receptors were prepared. The plasmids pMX-puromycin-MuIL-17RX-MuGCSFR were transfected into the BOSC23 retroviral packaging cells using FUGENE6 (Roche). Virus was collected after 48 hours and used to infect Baf/3 cells. Stable Baf/3 cells expressing the various IL-17R chimeric fusions were selected for with puromycin (1 μg/ml).

Members of the IL-17 receptor family, summarized in Table 2, were used to prepare chimeric receptors.

TABLE 2
Nomenclature of IL-17 receptor proteins.

| DNAX Cytokine Receptor Subunit (DCRS) | Alternate nomenclature |
|---|---|
| — | IL-17RA |
| DCRS6 | IL-17RB |
| DCRS7 | IL-17RC |
| DCRS8 | IL-17RD |
| DCRS9 | IL-17RE |
| DCRS10 | — |

FACS analysis was used to determine specific binding of the various ligands to the various receptors. Baf/3 or Baf/3 mouse IL17RX-mouse GCSFR cells were incubated with ligand containing cell supernatants for one hour on ice. The cells were then washed once in FACS buffer and resuspended in 10 μg/ml ANTI-FLAG® M2 antibody (Sigma, St. Louis, Mo.) for one hour on ice. Cells were washed once in FACS buffer and resuspended in 10 μl/ml anti-mouse IgG-PE (Jackson ImmunoResearch, West Grove, Pa.) for 30 minutes on ice. Cells were washed once in FACS buffer and resuspended in FACS buffer and 1 μl/ml propidium iodide (Roche Applied Science, Indianapolis, Ind.) and analyzed on a FACS Calibur (Becton Dickinson, San Jose, Calif.). The chimeric receptor cell based assay was used to visualize the binding of each of the IL-17 family ligands to the receptors by FACS.

IL-17C specifically bound to DCRS9 (IL-17RE), as shown in studies where IL-17RE was expressed from parental Baf/3 cells. IL-17C failed to bind to non-transfected parental Baf/3 cells or to cells transfected with IL-17RA, IL-17RB, IL-17RC, or IL-17RD.

Control studies demonstrated that IL-17F bound to the IL-17RC (a.k.a. DCRS7), that IL-17 bound IL-17RA and IL-17RC, and that IL-25 bound IL-17RB (a.k.a. DCRS6). Additionally, the IL-17RE transfected cells were negative for binding the ligands IL-17, IL-25, IL-17D, IL-17B and IL-17F.

IV. Expression of IL-17C and DCRS9 in Cells and Tissues.

Expression and distribution of IL-17C and DCRS9 in human cells and tissues were determined by Taqman® real time PCR (PE Applied Biosystems, Foster City, Calif.), where the results are relative to ubiquitin expression (Table 3). Ubiquitin expression is set to one (1.0).

TABLE 3
Expression of IL-17C and DCRS9.
Expression of human IL-17C

| | |
|---|---|
| Skin control | 0.0 |
| Skin psoriasis | 63.4 |
| Colon control | 0.0 |
| Colon Crohn's | 15.9 |
| Lung control | 0.0 |
| Lung hypersensitivity pneumonitis | 39.3 |
| Epithelial cell small airway untreated | 3.1 |
| Epithelial cell small airway activated IL-4 + IL-13 + TNFalpha | 12.1 |
| Epithelial cell keratinocyte, untreated | 1.6 |
| Epithelial cell keratinocyte, TNFa + IL-1 | 13.5 |
| Monocyte/PMBC resting | 29.8 |
| Monocyte/PBMC activated lipopolysaccharide (LPS) | 64.5 |
| Dendritic cell, resting ex monocytes 5d | 0.0 |
| Dendritic cell, activated LPS pool ex monocytes 5d 4 + 16 hours | 43.7 |
| Microvascular endothelial cell dermal untreated | 9.0 |
| Microvascular endothelial cell dermal activated TNFa + IL-1 | 40.3 |
| Microvascular endothelial cell lung untreated | 0.0 |
| Microvascular endothelial cell lung activated TNFa | 13.0 |

Expression of mouse IL-17C

| | |
|---|---|
| Mouse 129/SvEv colon untreated | 0.0 |
| Mouse IL-10 knockout colon | 54.4 |
| Mouse colon IBD model CD4⁺ CD45RBlo transfer | 68.8 |

Expression of human DCRS9

| | |
|---|---|
| Skin control | 0.0 |
| Skin psoriasis | 4.88 |
| Lung control | 42.0 |
| Lung hypersensitivity pneumonitis | 53.4 |
| Colon control | 0.0 |
| Colon Crohn's | 2.2 |
| Epithelial cell keratinocyte, untreated | 3.3 |
| Epithelial cell keratinocyte, activated, TNFa + IL-1 | 8.5 |

Expression of mouse DCRS9

| | |
|---|---|
| Mouse BALB/c lung untreated | 108.2 |
| Mouse lung infected Nippostrongylus | 168.2 |
| Mouse endothelial cell resting | 7.8 |
| Mouse endothelial cell activated TNFalpha 16 hour bEnd3 + | 14.0 |
| TNFalpha | |
| Mouse C57BL/6 aorta untreated | 16.1 |
| Mouse ApoE knockout aorta, atherosclerosis model, 5 | 106.3 |
| months age. | |

Additional studies relating to expression of members of the IL-17 family and IL-17 receptor family are as follows. Human multiple tissue (Cat#1, 2) and cancer cell line blots (Cat#7757-1), containing approximately 2 μtg of poly(A)⁺ RNA per lane, are purchased from Clontech (Palo Alto, Calif.). Probes are radiolabeled with [α-³²P] dATP, e.g., using the Amersham Rediprime random primer labeling kit (RPN1633). Prehybridization and hybridizations are performed, e.g., at 65° C. in 0.5 M Na₂HPO₄, 7% SDS, 0.5 M EDTA (pH 8.0). High stringency washes are conducted, e.g., at 65° C. with two initial washes in 2×SSC, 0.1% SDS for 40 min followed by a subsequent wash in 0.1×SSC, 0.1% SDS for 20 min. Membranes are then exposed at −70° C. to X-Ray film (Kodak) in the presence of intensifying screens. More detailed studies by cDNA library Southerns are performed with selected appropriate human DCRS clones to examine their expression in hemopoietic or other cell subsets.

Alternatively, two appropriate primers are selected from SEQ ID NOS: 1, 3, 5, 7, 9, 11, 13, 15, or 17. RT-PCR is used on an appropriate mRNA sample selected for the presence of message to produce a cDNA, e.g., a sample which expresses the gene.

Full length clones may be isolated by hybridization of cDNA libraries from appropriate tissues pre-selected by PCR signal. Northern blots can be performed.

Message for genes encoding DCRS will be assayed by appropriate technology, e.g., PCR, immunoassay, hybridization, or otherwise. Tissue and organ cDNA preparations are available, e.g., from Clontech, Mountain View, Calif. Identification of sources of natural expression is useful, as described. And the identification of functional receptor subunit pairings will allow for prediction of what cells express the combination of receptor subunits which will result in a physiological responsiveness to each of the cytokine ligands.

For mouse counterpart distribution, e.g., Southern Analysis can be performed: DNA (5 pg) from a primary amplified cDNA library was digested with appropriate restriction enzymes to release the inserts, run on a 1% agarose gel and transferred to a nylon membrane (Schleicher and Schuell, Keene, N.H.).

Samples for mouse mRNA isolation may include: resting mouse fibroblastic L cell line (C200); Braf:ER (Braf fusion to estrogen receptor) transfected cells, control (C201); T cells, TH1 polarized (Mel14 bright, CD4⁺ cells from spleen, polarized for 7 days with IFN-γ and anti IL-4; T200); T cells, TH2 polarized (Mel14 bright, CD4+ cells from spleen, polarized for 7 days with IL-4 and anti-IFN-γ; T201); T cells, highly TH1 polarized (see Openshaw, et al. (1995) J. Exp. Med. 182:1357-1367; activated with anti-CD3 for 2, 6, 16 h pooled; T202); T cells, highly TH2 polarized (see Openshaw, et al. (1995) J. Exp. Med. 182:1357-1367; activated with anti-CD3 for 2, 6, 16 h pooled; T203); CD44⁻ CD25⁺ pre T cells, sorted from thymus (T204); TH1 T cell clone D1.1, resting for 3 weeks after last stimulation with antigen (T205); TH1 T cell clone D1.1, 10 μg/ml ConA stimulated 15 h (T206); TH2 T cell clone CDC35, resting for 3 weeks after last stimulation with antigen (T207); TH2 T cell clone CDC35, 10 μg/ml ConA stimulated 15 h (T208); Mel14⁺ naive T cells from spleen, resting (T209); Mel14⁺ T cells, polarized to Th1 with IFN-γ/IL-12/anti-IL-4 for 6, 12, 24 h pooled (T210); Mel 14⁺ T cells, polarized to Th2 with IL-4/anti-IFN-γ for 6, 13, 24 h pooled (T211); unstimulated mature B cell leukemia cell line A20 (B200); unstimulated B cell line CH12 (B201); unstimulated large B cells from spleen (B202); B cells from total spleen, LPS activated (B203); metrizamide enriched dendritic cells from spleen, resting (D200); dendritic cells from bone marrow, resting (D201); monocyte cell line RAW 264.7 activated with LPS 4 h (M200); bone-marrow macrophages derived with GM and M-CSF (M201); macrophage cell line J774, resting (M202); macrophage cell line J774+LPS+anti-IL-10 at 0.5, 1, 3, 6, 12 h pooled (M203); macrophage cell line J774+LPS+IL-10 at 0.5, 1, 3, 5, 12 h pooled(M204); aerosol challenged mouse lung tissue, Th2 primers, aerosol OVA challenge 7, 14, 23 h pooled (see Garlisi, et al. (1995) Clinical Immunology and Immunopathology 75:75-83; X206); Nippostrongylus-infected lung tissue (see Coffman, et al. (1989) Science 245:308-310; X200); total adult lung, normal (O200); total lung, rag-1 (see Schwarz, et al. (1993) Immunodeficiency 4:249-252; O205); IL-10 K.O. spleen (see Kuhn, et al. (1991) Cell 75:263-274; X201); total adult spleen, normal (O201); total spleen, rag-1 (O207); IL-10 K.O. Peyer's patches (O202); total Peyer's patches, normal (O210); IL-10 K.O. mesenteric lymph nodes (X203); total mesenteric lymph nodes, normal (O211); IL-10 K.O. colon (X203); total colon, normal (O212); NOD mouse pancreas (see Makino, et al. (1980) Jikken Dobutsu 29:1-13; X205); total thymus, rag-1 (O208); total kidney, rag-1 (O209); total heart, rag-1 (O202); total brain, rag-1 (O203); total testes, rag-1 (O204); total liver, rag-1 (O206); rat normal joint tissue (O300); and rat arthritic joint tissue (X300).

Samples for human mRNA isolation may include, e.g.: peripheral blood mononuclear cells (monocytes, T cells, NK cells, granulocytes, B cells), resting (T100); peripheral blood mononuclear cells, activated with anti-CD3 for 2, 6, 12 h pooled (T101); T cell, TH0 clone Mot 72, resting (T102); T cell, TH0 clone Mot 72, activated with anti-CD28 and anti-CD3 for 3, 6, 12 h pooled (T103); T cell, TH0 clone Mot 72, anergic treated with specific peptide for 2, 7, 12 h pooled (T104); T cell, TH1 clone HY06, resting (T107); T cell, TH1 clone HY06, activated with anti-CD28 and anti-CD3 for 3, 6, 12 h pooled (TI08); T cell, TH1 clone HY06, anergic treated with specific peptide for 2, 6, 12 h pooled (T109); T cell, TH2 clone HY935, resting (T110); T cell, TH2 clone HY935, activated with anti-CD28 and anti-CD3 for 2, 7, 12 h pooled (T111); T cells CD4+CD45RO-T cells polarized 27 days in anti-CD28, IL-4, and anti IFN-γ, TH2 polarized, activated with anti-CD3 and anti-CD28 4 h (T116); T cell tumor lines Jurkat and Hut78, resting (T117); T cell clones, pooled AD130.2, Tc783.12, Tc783.13, Tc783.58, Tc782.69, resting (T118); T cell random γδ T cell clones, resting (T119); Splenocytes, resting (B100); Splenocytes, activated with anti-CD40 and IL-4 (B101); B cell EBV lines pooled WT49, RSB, JY, CVIR, 721.221, RM3, HSY, resting (B102); B cell line JY, activated with PMA and ionomycin for 1, 6 h pooled (B103); NK 20 clones pooled, resting (K100); NK 20 clones pooled, activated with PMA and ionomycin for 6 h (K101); NKL clone, derived from peripheral blood of LGL leukemia patient, IL-2 treated (K106); NK cytotoxic clone 640-A30-1, resting (K107); hematopoietic precursor line TF1, activated with PMA and ionomycin for 1, 6 h pooled (C100); U937 premonocytic line, resting (M100); U937 premonocytic line, activated with PMA and ionomycin for 1, 6 h pooled (M101); elutriated monocytes, activated with LPS, IFNγ, anti-IL-10 for 1, 2, 6, 12, 24 h pooled (M102); elutriated monocytes, activated with LPS, IFNγ, IL-10 for 1, 2, 6, 12, 24 h pooled (M103); elutriated monocytes, activated with LPS, IFNγ, anti-IL-10 for 4, 16 h pooled (M106); elutriated monocytes, activated with LPS, IFNγ, IL-10 for 4, 16. h pooled (M107); elutriated monocytes, activated LPS for 1 h (M108); elutriated monocytes, activated LPS for 6 h (M109); DC 70% CD1a+, from CD34+ GM-CSF, TNFα 12 days, resting (D101); DC 70% CD1a⁺, from CD34+ GM-CSF, TNFα 12 days, activated with PMA and ionomycin for 1 hr (D102); DC 70% CD1a⁺, from CD34⁺ GM-CSF, TNFα 12 days, activated with PMA and ionomycin for 6 hr (D103); DC 95% CD1a+, from CD34⁺ GM-CSF, TNFα 12 days FACS sorted, activated with PMA and ionomycin for 1, 6 h pooled (D104); DC 95% CD14⁺, ex CD34⁺ GM-CSF, TNFα 12 days FACS sorted, activated with PMA and ionomycin 1, 6 hr pooled (D105); DC CD1a+ CD86⁺, from CD34⁺ GM-CSF, TNFα 12 days FACS sorted, activated with PMA and ionomycin for 1, 6 h pooled (D106); DC from monocytes GM-CSF, IL-4 5 days, resting (D107); DC from monocytes GM-CSF, IL-4 5 days, resting (D108); DC from monocytes GM-CSF, IL-4 5 days, activated LPS 4, 16 h pooled (D109); DC from monocytes GM-CSF, IL-4 5 days, activated TNFα, monocyte supe for 4, 16 h pooled (D110); leiomyoma L11 benign tumor (X101); normal myometrium M5 (O115); malignant leiomyosarcoma GS1 (X103); lung fibroblast sarcoma line MRC5, activated with PMA and ionomycin for 1, 6 h pooled (C101); kidney epithelial carcinoma cell line CHA, activated with PMA and ionomycin for 1, 6 h pooled (C102); kidney fetal 28 wk male (O100); lung fetal 28 wk male (O101); liver fetal 28 wk male (O102); heart fetal 28 wk male (O103); brain fetal 28 wk male (O104); gallbladder fetal 28 wk male (O106); small intestine fetal 28 wk male (O107); adipose tissue fetal 28 wk male (O108); ovary fetal 25 wk female (O109); uterus fetal 25 wk female (O110); testes fetal 28 wk male (O111); spleen fetal 28 wk male (0112); adult placenta 28 wk (O113); and tonsil inflamed, from 12 year old (X100).

TaqMan® quantitative PCR techniques have shown the DCRS6, in both mouse and human, to be expressed on T cells, including thymocytes and CD4⁺ naive and differentiated (hDCRS6 is also expressed on dendritic cells), in gastrointestinal tissue, including stomach, intestine, colon and associated lymphoid tissue, e.g., Peyer's patches and mesenteric lymph nodes, and upregulated in inflammatory models of bowel disease, e.g., IL-10 KO mice. The. hDCRS7 was detected in both resting and activated dendritic cells, epithelial cells, and mucosal tissues, including GI and reproductive tracts. These data suggest that family members are expressed in mucosal tissues and immune system cell types, and/or in gastrointestinal, airway, and reproductive tract development.

As such, therapeutic indications include, e.g., short bowel syndrome, post chemo/radio-therapy or alcoholic recovery, combinations with ulcer treatments or arthritis medication, Th2 pregnancy skewing, stomach lining/tissue regeneration, loss of adsorptive surface conditions, etc. See, e.g., Yamada, et al. (eds.) (1999) Textbook of Gastroenterology; Yamada, et al. (eds.) (1999) Textbook and Atlas of Gastroenterology; Gore and Levine (2000) Textbook of Gastrointestinal Radiology; and (1987) Textbook of Pediatric Gastroenterology. Similar samples may isolated in other species for evaluation.

Primers specific for IL-17RA (Table 4) were designed and used in Taqman® quantitative PCR against various human libraries. IL-17A is highly expressed in innate immune myeloid cells including dendritic cells and monocytes. Expression is also detected in T cell libraries. These data demonstrate the receptor is expressed in immune cell types and may be regulated by activation conditions.

TABLE 4
IL-17RA library description.

| | CT for |
|---|---|
| | IL-17RA_H |
| DC ex monocytes GM-CSF, IL-4, resting | 16.97 |
| U937 premonocytic line, activated | 17.14 |
| DC ex monocytes GM-CSF, IL-4, resting | 17.53 |
| DC 70% CD1a+, ex CD34+ GM-CSF, TNFa, resting | 18.17 |
| monocytes, LPS, gIFN, anti-IL-10 | 18.27 |
| DC ex monocytes GM-CSF, IL-4, LPS activated 4 + | 18.51 |
| 16 hr | |
| DC ex monocytes GM-CSF, IL-4, monokine activated 4 + | 18.68 |
| 16 hr | |
| kidney epithelial carcinoma cell line CHA, activated | 18.69 |
| monocytes, LPS, 1 hr | 18.72 |
| monocytes, LPS, 6 hr | 18.72 |
| DC 70% CD1a+, ex CD34+ GM-CSF, TNFa, activated 1 hr | 18.91 |
| DC 70% CD1a+, ex CD34+ GM-CSF, TNFa, activated 6 hr | 18.94 |
| T cell, TH1 clone HY06, activated | 18.99 |
| lung fetal | 19.15 |
| T cell, TH1 clone HY06, resting | 19.18 |
| T cell, TH1 clone HY06, anergic | 19.23 |
| monocytes, LPS, gIFN, IL-10, 4 + 16 hr | 19.3 |
| spleen fetal | 19.51 |
| testes fetal | 19.7 |
| T cell, TH0 clone Mot 72, resting | 19.71 |
| T cell, TH0 clone Mot 72, resting | 19.84 |
| DC CD1a+ CD86+, ex CD34+ GM-CSF, TNFa, | 19.94 |
| activated 1 + 6 hr | |
| peripheral blood mononuclear cells, activated | 20.01 |
| hematopoietic precursor line TF1, activated | 20.07 |
| lung fibroblast sarcoma line MRC5, activated | 20.18 |
| Splenocytes, activated | 20.21 |
| T cell gd clones, resting | 20.27 |
| ovary fetal | 20.45 |
| T cells CD4+,TH2 polarized, activated | 20.57 |
| Splenocytes, resting | 20.6 |
| uterus fetal | 20.62 |
| DC 95% CD1a⁺, ex CD34+ GM-CSF, TNFa, | 20.94 |
| activated 1 + 6 hr | |
| epithelial cells, unstimulated | 20.96 |
| peripheral blood mononuclear cells, resting | 20.97 |
| adipose tissue fetal | 21.13 |
| B cell line JY, activated | 21.28 |
| monocytes, LPS, gIFN, IL-10 | 21.37 |
| placenta 28 wk | 21.38 |
| NK 20 clones pooled, activated | 21.55 |
| pool of two normal human lung samples | 21.63 |
| normal human thyroid | 21.65 |
| epithelial cells, IL-1b activated | 21.72 |
| normal human skin | 21.84 |
| T cell, TH0 clone Mot 72, anergic | 21.87 |
| small intestine fetal | 22.01 |
| CD28- T cell clone in pME | 22.08 |
| T cell, TH2 clone HY935, activated | 22.09 |
| T cell clones, pooled, resting | 22.29 |
| Hashimoto's thyroiditis thyroid sample | 22.3 |
| NK 20 clones pooled, resting | 22.4 |
| B cell EBV lines, resting | 22.45 |
| T cell, TH2 clone HY935, resting | 22.86 |
| T cell, TH0 clone Mot 72, activated | 23.3 |
| monocytes, LPS, gIFN, anti-IL-10, 4 + 16 hr | 23.39 |
| T cell lines Jurkat and Hut78, resting | 23.4 |
| T cell, TH0 clone Mot 72, activated | 23.56 |
| Pneumocystic carnii pneumonia lung sample | 24.05 |
| U937 premonocytic line, resting | 25.01 |
| pool of rheumatoid arthritis samples, human | 25.85 |
| pool of three heavy smoker human lung samples | 26.1 |
| DC 95% CD14⁺, ex CD34+ GM-CSF, | 32.69 |
| TNFa, activated 1 + 6 hr | |
| kidney fetal | 33.7 |
| liver fetal | 34.4 |
| NK cytotoxic clone, resting | 34.49 |
| tonsil inflammed | 35.02 |
| normal w.t. monkey lung | 35.45 |
| gallbladder fetal | 35.84 |
| TR1 T cell clone | 35.86 |
| allergic lung sample | 36.39 |
| Psoriasis patient skin sample | 36.44 |
| normal human colon | 37.34 |
| brain fetal | 37.35 |
| Ascaris-challenged monkey lung, 4 hr. | 37.75 |
| Ascaris-challenged monkey lung, 24 hr. | 40 |
| heart fetal | 40 |
| normal w.t. monkey colon | 40 |
| ulcerative colitis human colon sample | 40 |

Primers specific for DCRS6_H (Table 5) were designed and used in Taqman® quantitative PCR against various human libraries. DCRS6_H is expressed in innate immune myeloid cells including dendritic cells and monocytes. Expression is also detected in T cell libraries. These data demonstrate the receptor is expressed in immune cell types and may be regulated by activation conditions.

TABLE 5
DCRS6_H library description.

| | CT for |
|---|---|
| | DCRS6_H |
| T cell, TH0 clone Mot 72, resting | 15.54 |
| T cell, TH0 clone Mot 72, resting | 15.7 |
| DC ex monocytes GM-CSF, IL-4, resting | 17.84 |
| DC ex monocytes GM-CSF, IL-4, resting | 18.19 |
| DC ex monocytes GM-CSF, IL-4, LPS activated 4 + 16 hr | 18.3 |
| DC ex monocytes GM-CSF, IL-4, monokine activated 4 + | 18.3 |
| 16 hr | |
| T cell, TH1 clone HY06, resting | 18.43 |
| NK cytotoxic clone, resting | 18.53 |
| T cell clones, pooled, resting | 18.8 |
| T cell, TH1 clone HY06, activated | 19.03 |
| T cell, TH2 clone HY935, activated | 19.1 |
| TR1 T cell clone | 19.12 |
| T cells CD4+, TH2 polarized, activated | 20.06 |
| B cell EBV lines, resting | 20.3 |
| T cell, TH2 clone HY935, resting | 20.48 |
| kidney epithelial carcinoma cell line CHA, activated | 21.07 |
| T cell, TH1 clone HY06, anergic | 21.14 |
| normal human colon | 21.29 |
| NK 20 clones pooled, resting | 21.49 |
| T cell gd clones, resting | 21.58 |
| gallbladder fetal | 22.21 |
| kidney fetal | 22.79 |
| liver fetal | 22.8 |
| Pneumocystic carnii pneumonia lung sample | 23.06 |
| CD28− T cell clone in pME | 23.18 |
| T cell, TH0 clone Mot 72, anergic | 23.2 |
| ovary fetal | 23.51 |
| normal human thyroid | 24.03 |
| small intestine fetal | 24.13 |
| testes fetal | 24.82 |
| epithelial cells, IL-1b activated | 26.08 |
| pool of three heavy smoker human lung samples | 26.49 |
| placenta 28 wk | 26.56 |
| normal w.t. monkey lung | 28.65 |
| peripheral blood mononuclear cells, activated | 33.39 |
| Ascaris-challenged monkey lung, 4 hr. | 36.59 |
| spleen fetal | 38.43 |
| peripheral blood mononuclear cells, resting | 40 |
| T cell, TH0 clone Mot 72, activated | 40 |
| T cell lines Jurkat and Hut78, resting | 40 |
| Splenocytes, resting | 40 |
| Splenocytes, activated | 40 |
| B cell line JY, activated | 40 |
| NK 20 clones pooled, activated | 40 |
| hematopoietic precursor line TF1, activated | 40 |
| U937 premonocytic line, resting | 40 |
| U937 premonocytic line, activated | 40 |
| monocytes, LPS, gIFN, anti-IL-10 | 40 |
| monocytes, LPS, gIFN, IL-10 | 40 |
| monocytes, LPS, gIFN, anti-IL-10, 4 + 16 hr | 40 |
| monocytes, LPS, gIFN, IL-10, 4 + 16 hr | 40 |
| monocytes, LPS, 1 hr | 40 |
| monocytes, LPS, 6 hr | 40 |
| DC 70% CD1a⁺, ex CD34⁺ GM-CSF, TNFa, resting | 40 |
| DC 70% CD1a⁺, ex CD34⁺ GM-CSF, TNFa, activated 1 hr | 40 |
| DC 70% CD1a⁺, ex CD34⁺ GM-CSF, TNFa, activated 6 hr | 40 |
| DC 95% CD1a⁺, ex CD34⁺ GM-CSF, TNFa, activated 1 + | 40 |
| 6 hr | |
| DC 95% CD14⁺, ex CD34⁺ GM-CSF, TNFa, activated 1 + | 40 |
| 6 hr | |
| DC CD1a⁺ CD86⁺, ex CD34+ GM-CSF, TNFa, activated | 40 |
| 1 + 6 hr | |
| epithelial cells, unstimulated | 40 |
| lung fibroblast sarcoma line MRC5, activated | 40 |
| Ascaris-challenged monkey lung, 24 hr. | 40 |
| pool of two normal human lung samples | 40 |
| allergic lung sample | 40 |
| normal w.t. monkey colon | 40 |
| ulcerative colitis human colon sample | 40 |
| Hashimoto's thyroiditis thyroid sample | 40 |
| pool of rheumatoid arthritis samples, human | 40 |
| normal human skin | 40 |
| Psoriasis patient skin sample | 40 |
| tonsil inflammed | 40 |
| lung fetal | 40 |
| heart fetal | 40 |
| brain fetal | 40 |
| adipose tissue fetal | 40 |
| uterus fetal | 40 |
| T cell, TH0 clone Mot 72, activated | 40 |

Primers specific for DCRS7_H (Table 6) were designed and used in Taqman® quantitative PCR against various human libraries. DCRS6_H is expressed in innate immune myeloid cells including dendritic cells and monocytes. Expression is also detected in T cell libraries. These data demonstrate the receptor is expressed in immune cell types and may be regulated by activation conditions.

TABLE 6
DCRS7_H library description

| | CT for |
|---|---|
| | DCRS7_H |
| fetal uterus | 19.05 |
| DC mix | 19.34 |
| fetal small intestine | 19.46 |
| fetal ovary | 19.68 |
| fetal testes | 19.75 |
| fetal lung | 20.04 |
| CHA | 20.24 |
| normal thyroid | 20.32 |
| DC/GM/IL-4 | 20.52 |
| fetal spleen | 20.86 |
| normal lung | 20.94 |
| TF1 | 21 |
| allergic lung #19 | 21.02 |
| Psoriasis skin | 21.07 |
| fetal liver | 21.15 |
| MRC5 | 21.15 |
| 24 hr. Ascaris lung | 21.17 |
| hi dose IL-4 lung | 21.23 |
| CD1a⁺ 95% | 21.32 |
| Hashimotos thyroiditis | 21.35 |
| Crohns colon 4003197A | 21.35 |
| normal lung pool | 21.36 |
| 70% DC resting | 21.42 |
| fetal kidney | 21.58 |
| adult placenta | 21.68 |
| lung 121897-1 | 21.8 |
| Pneumocystis carnii lung #20 | 21.81 |
| A549 unstim. | 21.89 |
| normal colon #22 | 21.94 |
| 18 hr. Ascaris lung | 22.09 |
| normal skin | 22.1 |
| Crohns colon 9609C144 | 22.13 |
| fetal adipose tissue | 22.35 |
| D6 | 22.39 |
| DC resting CD34-derived | 22.45 |
| DC TNF/TGFb act CD34-der. | 22.54 |
| fetal brain | 22.9 |
| DC CD40L activ. mono-deriv. | 22.91 |
| Crohns colon 403242A | 22.91 |
| ulcerative colitis colon #26 | 23 |
| RA synovium pool | 23.06 |
| A549 activated | 23.06 |
| mono + IL-10 | 23.42 |
| DC LPS | 23.49 |
| Mot 72 activated | 23.66 |
| CD1a⁺ CD86⁺ | 23.86 |
| HY06 resting | 23.87 |
| U937 activated | 23.97 |
| inflammed tonsil | 23.97 |
| D1 | 24.06 |
| M1 | 24.17 |
| CD14⁺ 95% | 24.21 |
| lung 080698-2 | 24.28 |
| 4 hr. Ascaris lung | 24.37 |
| Jurkat activated pSPORT | 24.42 |
| DC resting mono-derived | 24.48 |
| HY06 activated | 24.54 |
| C+ | 24.64 |
| Splenocytes resting | 24.65 |
| U937/CD004 resting | 24.96 |
| PBMC resting | 25.8 |
| Mot 72 resting | 25.91 |
| mono + anti-IL-10 | 26.14 |
| NK pool | 26.99 |
| HY06 anti-peptide | 27.34 |
| mast cell pME | 27.38 |
| Tc gamma delta | 28.14 |
| TC1080 CD28− pMET7 | 31.05 |
| PBMC activated | 31.89 |
| NK non cytotox. | 32.3 |
| RV-C30 TR1 pMET7 | 32.5 |
| Bc | 33.72 |
| C− | 33.8 |
| Splenocytes activated | 34.7 |
| JY | 35.05 |
| NK cytotox. | 36.44 |
| NKL/IL-2 | 37.59 |
| HY935 resting | 37.6 |
| NK pool activated | 38.15 |
| Mot 72 anti-peptide | 38.87 |
| fetal heart | 40.92 |
| B21 resting | 42.05 |
| Jurkat resting pSPORT | 42.8 |
| B21 activated | 43.09 |
| NKA6 pSPORT | 44.85 |
| HY935 activated | 45 |
| M6 | 45 |

Primers specific for DCRS9_H (Table 7) were designed and used in Taqman® quantitative PCR against various human libraries. DCRS9_H is expressed in innate immune myeloid cells including dendritic cells and monocytes. Expression is also detected in T cell libraries. These data demonstrate the receptor is expressed in immune cell types and may be regulated by activation conditions.

TABLE 7
DCRS9_H library description.

| | CT for |
|---|---|
| | DCRS9_H |
| HY06 resting | 22.35 |
| fetal lung | 22.63 |
| HY06 anti-peptide | 22.72 |
| HY06 activated | 22.96 |
| U937/CD004 resting | 24.16 |
| fetal small intestine | 24.94 |
| JY | 25.04 |
| Mot 72 resting | 25.12 |
| Jurkat activated pSPORT | 25.2 |
| RV-C30 TR1 pMET7 | 26.51 |
| fetal kidney | 26.76 |
| MRC5 | 27.2 |
| Psoriasis skin | 27.3 |
| Tc gamma delta | 27.37 |
| Crohn's colon 4003197A | 27.44 |
| fetal spleen | 27.72 |
| normal lung | 27.83 |
| Hashimotos thyroiditis | 28.03 |
| B21 resting | 28.32 |
| TF1 | 28.39 |
| NK cytotox. | 28.44 |
| TC1080 CD28− pMET7 | 28.61 |
| Pneumocystis carnii lung #20 | 29.05 |
| U937 activated | 29.06 |
| HY935 resting | 29.09 |
| CD1a+ 95% | 29.13 |
| B21 activated | 29.2 |
| Mot 72 activated | 29.21 |
| fetal testes | 29.27 |
| lung 080698-2 | 29.32 |
| Jurkat resting pSPORT | 29.38 |
| CD14⁺ 95% | 29.38 |
| normal thyroid | 29.53 |
| Mot 72 anti-peptide | 29.65 |
| Splenocytes resting | 29.85 |
| Crohns colon 9609C144 | 30.28 |
| lung 121897-1 | 30.37 |
| 24 hr. Ascaris lung | 30.59 |
| hi dose IL-4 lung | 30.8 |
| CD1a⁺ CD86+ | 31.42 |
| normal skin | 31.73 |
| fetal uterus | 31.79 |
| PBMC activated | 31.82 |
| inflammed tonsil | 31.98 |
| fetal brain | 32.21 |
| RA synovium pool | 32.77 |
| allergic lung #19 | 33.18 |
| 18 hr. Ascaris lung | 33.42 |
| adult placenta | 33.43 |
| normal lung pool | 33.45 |
| Crohn's colon 403242A | 33.52 |
| NK pool | 33.72 |
| HY935 activated | 33.75 |
| DC/GM/IL-4 | 34.28 |
| DC resting mono-derived | 34.57 |
| fetal ovary | 35.06 |
| fetal adipose tissue | 35.07 |
| CHA | 35.2 |
| PBMC resting | 35.95 |
| Bc | 36.19 |
| A549 unstim. | 36.4 |
| fetal heart | 36.87 |
| ulcerative colitis colon #26 | 37.83 |
| C− | 38.32 |
| 4 hr. Ascaris lung | 40.2 |
| D6 | 40.62 |
| C+ | 44.38 |
| A549 activated | 44.58 |
| Splenocytes activated | 45 |
| NK pool activated | 45 |
| NKA6 pSPORT | 45 |
| NKL/IL-2 | 45 |
| NK non cytotox. | 45 |
| mono + anti-IL-10 | 45 |
| mono + IL-10 | 45 |
| M1 | 45 |
| M6 | 45 |
| 70% DC resting | 45 |
| D1 | 45 |
| DC LPS | 45 |
| DC mix | 45 |
| fetal liver | 45 |
| mast cell pME | 45 |
| DC CD40L activ. mono-deriv. | 45 |
| DC resting CD34-derived | 45 |
| DC TNF/TGFb act CD34-der. | 45 |
| normal colon #22 | 45 |

V. Cloning of Full-Length cDNAs; Chromosomal Localization

PCR primers derived from the sequences are used to probe a human cDNA library. Sequences may be derived, e.g., from SEQ ID NOS: 1, 3, 5, 7, 9, 11, 13, 15, or 17, preferably those adjacent the ends of sequences. Full length cDNAs for primate, rodent, or other species DCRS8 are cloned, e.g., by DNA hybridization screening of λgt10 phage. PCR reactions are conducted using T. aquaticus Taqplus DNA polymerase (Stratagene) under appropriate conditions. Extending partial length cDNA clones is typically routine.

Chromosome spreads are prepared. In situ hybridization is performed on chromosome preparations obtained from phytohemagglutinin-stimulated human lymphocytes cultured for 72 h. 5-bromodeoxyuridine was added for the final seven hours of culture (60 μg/ml of medium), to ensure a posthybridization chromosomal banding of good quality.

A PCR fragment, amplified with the help of primers, is cloned into an appropriate vector. The vector is labeled by nick-translation with ³H. The radiolabeled probe is hybridized to metaphase spreads at final concentration of 200 ng/ml of hybridization solution as described, e.g., in Mattei, et al. (1985) Hum. Genet. 69:327-331.

After coating with nuclear track emulsion (Kodak NTB_{2®}), slides are exposed. To avoid any slipping of silver grains during the banding procedure, chromosome spreads are first stained with buffered Giemsa solution and metaphase photographed. R-banding is then performed by the fluorochrome-photolysis-Giemsa (FPG) method and metaphases rephotographed before analysis. Similar appropriate methods are used for other species.

VI. Cloning of Species Counterparts

Various strategies are used to obtain species counterparts of the DCRSs, preferably from other primates or rodents. One method is by cross hybridization using closely related species DNA probes. It may be useful to go into evolutionarily similar species as intermediate steps. Another method is by using specific PCR primers based on the identification of blocks of similarity or difference between genes, e.g., areas of highly conserved or nonconserved polypeptide or nucleotide sequence. Sequence database searches may identify species counterparts.

VII. Production of Mammalian Protein

An appropriate, e.g., GST, fusion construct is engineered for expression, e.g., in E. coli. For example, a mouse IGIF pGex plasmid is constructed and transformed into E. coli. Freshly transformed cells are grown, e.g., in LB medium containing 50 μg/ml ampicillin and induced with IPTG (Sigma, St. Louis, Mo.). After overnight induction, the bacteria are harvested and the pellets containing the appropriate protein are isolated. The pellets are homogenized, e.g., in TE buffer (50 mM Tris-base pH 8.0, 10 mM EDTA and 2 mM pefabloc) in 2 liters. This material is passed through a microfluidizer (Microfluidics, Newton, Mass.) three times. The fluidized supernatant is spun down on a Sorvall GS-3 rotor for 1 h at 13,000 rpm. The resulting supernatant containing the cytokine receptor protein is filtered and passed over a glutathione-Sepharose® column equilibrated in 50 mM Tris-base pH 8.0. Fractions containing the IL-17C or DCRS9-GST fusion protein are pooled and cleaved, e.g., with thrombin (Enzyme Research Laboratories, Inc., South Bend, Ind.). The cleaved pool is then passed over a Q-Sepharose® column equilibrated in 50 mM Tris-base. Fractions containing IL-17C or DCRS9 are pooled and diluted in cold distilled H₂O, to lower the conductivity, and passed back over a fresh Q-Sepharose® column, alone or in succession with an immunoaffinity antibody column. Fractions containing the IL-17C or DCRS9 protein are pooled, aliquoted, and stored in the −70° C. freezer.

Comparison of the CD spectrum with cytokine receptor protein may suggest that the protein is correctly folded. See Hazuda, et al. (1969) J. Biol. Chem. 264:1689-1693.

VIII. Preparation of Specific Antibodies

Inbred Balb/c mice are immunized intraperitoneally with recombinant forms of the protein, e.g., purified IL-17C or DCRS9 or stable transfected NIH-3T3 cells. Animals are boosted at appropriate time points with protein, with or without additional adjuvant, to further stimulate antibody production. Serum is collected, or hybridomas produced with harvested spleens.

Alternatively, Balb/c mice are immunized with cells transformed with the gene or fragments thereof, either endogenous or exogenous cells, or with isolated membranes enriched for expression of the antigen. Serum is collected at the appropriate time, typically after numerous further administrations. Various gene therapy techniques may be useful, e.g., in producing protein in situ, for generating an immune response. Serum or antibody preparations may be cross-absorbed or immunoselected to prepare substantially purified antibodies of defined specificity and high affinity.

Monoclonal antibodies may be made. For example, splenocytes are fused with an appropriate fusion partner and hybridomas are selected in growth medium by standard procedures. Hybridoma supernatants are screened for the presence of antibodies which bind to the L-17C or DCRS9, e.g., by ELISA or other assay. Antibodies which specifically recognize specific IL-17C or DCRS9 embodiments may also be selected or prepared.

In another method, synthetic peptides or purified protein are presented to an immune system to generate monoclonal or polyclonal antibodies. See, e.g., Coligan (ed.) (1991) Current Protocols in Immunology, Wiley/Greene; and Harlow and Lane, supra. In appropriate situations, the binding reagent is either labeled as described above, e.g., fluorescence or otherwise, or immobilized to a substrate for panning methods. Nucleic acids may also be introduced into cells in an animal to produce the antigen, which serves to elicit an immune response. See, e.g., Wang, et al. (1993) Proc. Natl. Acad. Sci. USA 90:4156-4160; Barry, et al. (1994) Bio Techniques 16:616-619; and Xiang, et al. (1995) Immunity 2: 129-135.

IX. Production of Fusion Proteins

Various fusion constructs are made with IL-17C (SEQ ID NO:24) or DCRS9 (SEQ ID NO:12). A portion of the appropriate gene is fused to an epitope tag, e.g., a FLAG tag, or to a two hybrid system construct. See, e.g., Fields and Song (1989) Nature 340:245-246.

The epitope tag may be used in an expression cloning procedure with detection with anti-FLAG antibodies to detect a binding partner, e.g., ligand for the respective cytokine receptor. The two hybrid system may also be used to isolate proteins which specifically bind to the receptor subunit.

X. Structure Activity Relationship

Information on the criticality of particular residues is determined using standard procedures and analysis. Standard mutagenesis analysis is performed, e.g., by generating many different variants at determined positions, e.g., at the positions identified above, and evaluating biological activities of the variants. This may be performed to the extent of determining positions which modify activity, or to focus on specific positions to determine the residues which can be substituted to either retain, block, or modulate biological activity.

Alternatively, analysis of natural variants can indicate what positions tolerate natural mutations. This may result from populational analysis of variation among individuals, or across strains or species. Samples from selected individuals are analyzed, e.g., by PCR analysis and sequencing. This allows evaluation of population polymorphisms.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of modulating an activity of a cell comprising contacting the cell with an agonist or antagonist of DCRS9 (SEQ ID NOs:11 or 12) or of IL-17C (SEQ ID NOs:23 or 24), wherein the cell modulates:
a) psoriasis;
b) inflammatory bowel disorder (IBD);
c) interstitial lung-disorder;
d) asthma or allergy; or
e) atherosclerosis.

2. The method of claim 1, wherein the cell is:
a) a monocyte or a macrophage;
b) a dendritic cell;
c) an epithelial cell;
d) an endothelial cell; or
e) a keratinocyte.

3. The method of claim 1, wherein the activity is cytokine expression.

4. The method of claim 1, wherein the agonist or antagonist specifically binds to a polypeptide or nucleic acid of:
a) DCRS9 (SEQ ID NOs:11 or 12); or
b) IL-17C (SEQ ID NOs:23 or 24).

5. The method of claim 1, wherein the agonist or antagonist comprises a binding composition derived from an antigen binding site of an antibody that specifically binds to a polypeptide of:
a) DCRS9 (SEQ ID NO:12); or
b) IL-17C (SEQ ID NO:24).

6. The method of claim 5, wherein the binding composition comprises:
a) a polyclonal antibody;
b) a monoclonal antibody;
c) a humanized antibody;
d) an Fab, Fv, or F(ab′)₂ fragment; or
e) a peptide mimetic of an antibody.

7. The method of claim 1, wherein the antagonist comprises a soluble receptor derived from DCRS9 (SEQ ID NO:12).

8. The method of claim 1, wherein the antagonist comprises an:
a) anti-sense nucleic acid; or
b) interference RNA nucleic acid.

9. A method of treating a subject suffering from a disorder comprising:
a) psoriasis;
b) inflammatory bowel disorder (IBD);
c) interstitial lung disorder;
d) asthma or allergy; or
e) atherosclerosis;
by administering an effective amount of an agonist or antagonist of DCRS9 (SEQ ID NOs:11 or 12) or of IL-17C (SEQ ID NOs:23 or 24).

10. The method of claim 9, wherein the disorder is mediated by:
a) monocytes or macrophages;
b) dendritic cells;
c) epithelial cells;
d) endothelial cells; or
e) keratinocytes.

11. The method of claim 9, wherein the interstitial lung disorder comprises:
a) idiopathic pulmonary fibrosis;
b) eosinophilic granuloma; or
c) hypersensitivity pneumonitis.

12. The method of claim 9, wherein the IBD comprises:
a) Crohn's disease; or
b) ulcerative colitis.

13. The method of claim 9, wherein the agonist or antagonist specifically binds to a polypeptide or nucleic acid of:
a) DCRS9 (SEQ ID NOs:11 or 12); or
b) IL-17C (SEQ ID NOs:23 or 24).

14. The method of claim 9, wherein the antagonist is a binding composition derived from an antigen binding site of an antibody that specifically binds to a polypeptide of:
a) DCRS9 (SEQ ID NO:12); or
b) IL-17C (SEQ ID NO:24).

15. The method of claim 14, wherein the binding composition comprises:
a) a polyclonal antibody;
b) a monoclonal antibody;
c) a humanized antibody;
d) an Fab, Fv, or F(ab′)₂ fragment; or
e) a peptide mimetic of an antibody.

16. The method of claim 9, wherein the agonist or antagonist is a soluble receptor derived from DCRS9 (SEQ ID NO:24).

17. The method of claim 9, wherein the antagonist comprises an:
a) anti-sense nucleic acid; or
b) interference RNA nucleic acid.

18. A method of diagnosing a disorder of the method of claim 9 comprising contacting a sample from a test subject with a binding composition that specifically binds to a polypeptide or nucleic acid of:
a) DCRS9 (SEQ ID NOs:11 or 12); or
b) IL-17C (SEQ ID NOs:23 or 24).

19. The method of claim 18, wherein the sample is derived from a tissue, cell, or biological fluid.

20. The method of claim 18, further comprising:
a) contacting a sample from a normal subject or a control source with the binding composition; and
b) comparing the binding to the test subject with the binding to the normal subject or control source.
